(19) United States
(12) Patent Application Publication
(10) Pub. No.: US 2019/0352741 A1
(43) Pub. Date: Nov. 21, 2019

(54) METHOD OF DETECTION AND EXTRACTING METALS FROM ORE-BEARING SLURRY

(71) Applicant: OUTCOME INTERNATIONAL INC., Bridgetown (BB)

(72) Inventor: Bernhard Peter BUDACH, JR., Huancayo (PE)

(21) Appl. No.: 16/476,092

(22) PCT Filed: Dec. 22, 2017
(86) PCT No.: PCT/IB2017/058371

§ 371 Date: Jul. 4, 2019

(30) Foreign Application Priority Data
Jan. 6, 2017 (CA) 2,953,756

(51) Int. Cl. C22B 3/00; C22B 3/20; G01N 27/08; G01N 33/20

(52) U.S. Cl. CPC C22B 11/042; G01N 33/20; G01N 27/08; C22B 3/20

(57) ABSTRACT

Method and apparatus are provided to select precious metals slurries of ore and water. Slurry is directed to pass over detectors, each comprising a pair of low voltage electrodes. The electrodes are spaced apart to form a detection gap. A slurry sample, having metals therein, is received at the gap. Metals at the gap generate a signal to trigger actuation to shunt the sample slurry and metals therein to a collection stream. Each collection stream can be processed in a similar, yet subsequent, refinement stage. Remaining slurry passes by the detector for further processing or as waste. One or more detectors are provided and, preferably, an array of detectors is provided in series and in stages, for collection efficiency. Each series of detectors can be provided in parallel arrangements for increased collection capacity. Detectors can be housed in modular sampling units for shipping and assembly efficiency.

### FIELD

Embodiments disclosed herein generally relate generally to methods for the detection and extraction of metals, from a slurried ore. More particularly electrodes fit to one or more rotary apparatus are spaced along a stream of slurry for the detection of metals, and diversion thereof, for recovery.

### BACKGROUND

Conventional processes to capture precious metals like gold, silver and platinum from minerals in a slurry of mud and water are typically handled by large machines and equipment. Such processes perform separation using gravitational settling and employ significant manpower. Such processes are also known as gold washing.

Such manual selection processes are not generally able to select small particles containing metals. Further, such conventional equipment is not adequate to select precious metals from rocks or ore containing auriferous metals.

Further, there are also chemical processes known for the separation of gold from auriferous metals. Such processes are less than optimal for recovery of gold and other precious metals from ore and, further, the chemicals and waste are a hazard to both personnel and the environment.

### SUMMARY

Method and apparatus are provided to select metals of interest, such as precious metals like gold, silver and platinum, from a slurry of ore and water. Metals of interest further include other metals that are of commercial value and that also conduct an electrical current. An objective of the embodiments disclosed herein is to provide an industrial method to select those metals through process and equipment that use the electrical properties of the subject metals. Accordingly, an effective and specific selection and recovery of metals from a slurry can be achieved without the danger or compromise to the environment associated with the prior technologies.

In an embodiment, ore or earth and rock are prepared as a mud or slurry which contains metals. In the detection and recovery portion of the method and apparatus disclosed herein, slurry is directed to pass along the channel and over at least one detector in series, each detector comprising a pair of electrodes. The slurry is typically flowing in an open top trough or channel. The detector is located in the channel in contact with the feedstream of slurry. The two electrodes of the detector are spaced apart to form a detection gap. A slurry sample of the slurry stream, having metals therein, is received at the gap. Metals detected at the gap generate a signal that triggers actuation of the detector to shunt or redirect the sample slurry and metals therein to a collection stream. Remaining slurry passes by the detector for further processing or to be collected as waste.

One or more detectors are provided and, preferably, an array of detectors are provided in series, for collection efficiency. Each series of detectors can be provided in parallel arrangements for increased collection capacity.

In embodiments each detector is a rotary sampler having at least one pair of electrodes forming the gap. The rotary sampler is situated in the slurry stream and can be actuated between a sampling position and a dump positions. Upon detection of metals at the gap, the rotary sampler is actuated from the sampling position to the dump position to direct the slurry sample from the main slurry stream and dump the slurry sample into the collection stream. The rotary sample can rotational on an axis which in one embodiment is a generally horizontal axis for moving a slurry sample from above a boundary, such as a channel bottom, to below the boundary. Other samplers, such as a pan-type sampler can have a generally vertical axis for shifting the slurry sample laterally through a boundary, such as a channel wall. In either case or other rotary samplers, the slurry sample is moved through a boundary wall from the feed stream to the collection stream.

The actuation of each rotary sampler is rotationally indexed from the sampling to the dumping position as each sample slurry having metals is detected, the sample slurry being directed to collection. With continuous metals detected in the slurry, the rotational indexing can be substantially continuous as to be virtually imperceptible to the human eye as individual movement.

In embodiments, each slurry sampler is a roller having an axis extending transversely across the feed stream of slurry flowing in a feed channel. The roller can be located along the bottom of the feed channel and inset about one halfway into the channel for exposure of the upper portion of the sampler to the slurry above the bottom of the channel.

The slurry flows over the roller, the roller sealing the bottom of the feed channel so that the feed stream of slurry continues therealong until such time as the roller is actuated to direct a slurry sample containing metals through the bottom and into a collection stream below the feed channel. Each roller can have more than one pair of electrodes located and spaced circumferentially about the roller. Further, each pair of electrodes can extend substantially fully along the roller axis or only partially there along.

Each roller can be generally cylindrical for ease of sealing in the bottom of the channel during actuation. Each electrode pair can be recessed radially within a groove or recess along the roller for forming a sampling volume. A slurry sample having metals therein and entering the recess, will actuate the roller to rotationally index, moving the recess and slurry sample from the slurry feed stream to the collection stream. After dumping the slurry sample, the recess is returned to the feed stream, or during dumping of a first recess, another recess is simultaneously positioned in the feed stream to repeat the sampling and detection process.

The selected content of each sampler, the slurry sample containing metals, is dumped, falls or is otherwise directed from one feed channel to a collection channel, each subsequent collection channel forming the feed channel for a next stage of sampling. The same selection methodology is applied to each successive collected sample until a desired concentrated or rich amount of high grade metals results. A plurality of staged of selection processes can follow until a very concentrated and rich amount of metals are recovered. Further, the apparatus for staged selection can be modularized, providing multistage units in a vertically descending manner, each stage pre-fit with detectors, the length of each stage's channel being readily increased by end-to-end coupling of additional modules.

At each stage, the collection channel is fit with slurry samplers, but each sampler can be adapted to the character of the successively higher concentration collected sample from the previous stage. For example, the sampler can be fit with more metal detectors, such as a large number of recesses about the circumference of a rotary roller-type sampler, or provided a roller of successively smaller diameter, or providing a larger concentration of samplers in series. Through this extraction and recovery process, and apparatus used therein, recovery speed is increased over the traditional gravity separation methodologies for gold, silver and other metals.

### BRIEF DESCRIPTION OF THE DRAWINGS

FIG. 1A is a flow schematic of a feed stream of ore slurry passing over a rotary sampler with a sample being diverted to a collection stream below;

FIG. 1B is a flow schematic of the feed stream of ore slurry of FIG. 1A passing over a series of rotary samplers with three of four samples, having metals therein, being diverted to a collection stream below;

FIG. 1C is a flow schematic of the feed stream of ore slurry of FIG. 1A passing over a series of rotary samplers with three of four samples, having metals therein, being diverted to a collection stream below, the collection stream passing over a series of rotary samplers for further detection and concentration of metals detected therein;

FIG. 2 is a flow schematic of a feed stream of ore slurry with four stages of metals concentration, each stream having a portion of the stream containing metals diverted to the next stage and the balance continuing to waste or secondary processing;

FIG. 3 is a partial side cross-sectional view of one rotary sampler sampling a feed stream flowing there over;

FIGS. 4A, 4B and 4C are a series off partial, side, cross-sectional views of a rotary sampler in three sequential stages of operation, namely showing a rotary sampler sampling a feed stream flowing there over, the sampler having detected metals therein dumping its sample for collection, and resetting to re-enter the feed stream to resume sampling as shown in FIG. 4A;

FIG. 5 is a flow chart of the process according to FIG. 2 and FIGS. 4A to 4C for illustrating the sequence of sampling, detecting, dumping and processing the dumped and collected stream in a subsequent stage;

FIG. 6A is a combined end perspective view of a rotary sampler and a flow sheet depicting operation of the stepper motor based on the electrode signals;

FIG. 6B illustrates a perspective view of one embodiment of a rotary sampler comprising a generally cylindrical roller having a longitudinal and right-angled recess formed there along, the base of the recess having a pair of electrodes extending therealong;

FIG. 7 is a perspective view of a feed stream channel with a metal detector and diverting gate for diverting the feed stream, absent metals, from further processing by the rotary sampler or samplers;

FIG. 8A is a perspective view of a portion of a channel with several embodiments of samples, including linear and rotary samplers;

FIG. 8B is a perspective view of a rotary table sample, with sampler recesses rotationally aligned outside the channel with apertures leading to the collection stream;

FIG. 8C is an end, cross-sectional view of the rotary table sampler for FIG. 8B illustrating the apertures leading to the collection stream;

FIG. 9 is a flow chart of a process sampling the feed stream slurry with a rotary sampler with a stepper motor and having a jam recovery sequence;

FIG. 10 is a flow chart of a process for monitoring signals from a pair of electrodes for determining if a sample has sufficient metal for dumping to the collection stream;

FIG. 11A is a perspective view of an embodiment of a single stage system for the serial detection and recovery for metals from a feedstream of slurried ore;

FIG. 11B is a perspective and exploded view of the system of FIG. 11A;

FIG. 11C is an exploded perspective view of the channel of FIG. 11A, with each rotary sampler also exploded into components;

FIG. 12A is a perspective view of an embodiment of a three-stage system having dual, parallel channels and detection systems, each channel of the first stage having serial detection and recovery for metals from a feedstream of slurried ore, for diversion to a collection stream in a single channel of a second stage, for diversion to a collection stream in a single channel of a third stage;

FIG. 12B is a perspective and exploded view of the first and second stages of the system of FIG. 12A;

FIG. 13 is a perspective view of an embodiment of a single-stage system having four, parallel channels and detection systems, each channel of the first stage having serial detection and recovery for metals from a feed stream of slurry ore, for diversion to a collection stream;

FIGS. 14A through 14I are end views of a variety of rotary sampler rollers having varied recesses formed therein, FIGS. 14A through 14F, 14I having generally rectangular electrodes, FIG. 14G having triangular electrodes, and further,

FIG. 14A having a right angle, single recess;

FIG. 14B having two opposed and generally trapezoidal recesses;

FIG. 14C having three generally trapezoidal recesses at 120 degrees;

FIG. 14D having six generally trapezoidal and equally spaced recesses about the circumference;

FIG. 14E having eight radially deep, generally trapezoidal and equally-spaced recesses about the circumference;

FIG. 14F having eight radially shallow, generally trapezoidal and equally-spaced recesses about the circumference;

FIG. 14G having eight generally trapezoidal and equally-spaced recesses about the circumference and triangular electrodes.

FIG. 14H having a triangular recess;

FIG. 14I having a polygonal recess having a plurality of electrode pairs and a knife edge on the leading edge for clean passage of the retained sample through the channel port to the next stage;

FIG. 15 is an exploded view of a rotary roller sampler assembly; having a step motor connect to driving clutch components, driven clutch components connected to the roller, and the bearing supports to the apparatus structure

FIG. 16A is a cross-sectional side view of a roller-type sample, having a slip clutch;

FIG. 16B is a partial cross-sectional side view of the slip clutch to roller connection according to FIG. 16A;

FIG. 17A is an end, exploded view of an electrode installation and replacement method for a roller-type sampler;

FIG. 17B is a close up perspective view of electrical socket connections for the replaceable electrodes of FIG. 17A;

FIG. 18A is an axially exploded view of the interface plates of the slip-clutch of FIG. 15, the driving portion of the slip-clutch comprising a plate having a driving face with semi-spherical recesses therein, and the driven portion having a driven face having semi-spherical protrusions extending therefrom, corresponding in number and circumferential location to the recesses in the driving face. The driving face is axially biased with a spring to forcibly engages the driven face with the protrusions within the recesses to permit co-rotation when the roller is freely rotatable, and to permit the driving face to withdrawn and skip over the protrusions if the roller ceases to rotate;

FIGS. 18B and 18C are end and cross-sectional views respectively of an assembled slip clutch according to FIG. 15, the driving and driven plates in engagement;

FIG. 19 is a perspective and exploded view of the roller end plate and rotary electrical connection system for four sets of electrodes, the electrodes shown in isolation from the supporting roller, each of the four sets of electrodes comprises three electrodes, one ground and two positives providing two detection gaps with three electrodes, two circular electrical contacts are provided, for ground and one for a positive terminal. The roller end plate is fit with electrical contacts for rotationally aligning with two non-rotation contacts that align at each of the four pole positions;

FIG. 20 is a perspective and exploded view of the roller end plate and electrodes of FIG. 19 coupled axially and fit with the slip-clutch system, the roller outline shown in dotted lines;

FIG. 21 is a perspective view of an embodiment of a step-wise modular unit with call outs to various components;

FIG. 22 is an exploded view of the step-wise modular unit of FIG. 21 with two additional units that can be connected for additional sampling capability;

FIG. 23 is a metal recovery system according to another embodiment a slurry mixing unit, a distribution system for parallel units, and a modular system of sampling units for adding units for parallel stream processing, and for lengthening the channels as needed for the feedstream;

FIG. 24A illustrates a perspective view of the mixer of FIG. 23;

FIG. 24B illustrates a top view of a distribution system for initially determining if the slurry should be directed to each of five selection units or redirected to waste, and if directed for selection, dividing the feedstream in distributor tank between five units;

FIG. 24C illustrates a perspective view of a distributor tank for the distributor of FIG. 24B;

FIG. 24D illustrates a perspective view of one module for forming one or more selection units of FIG. 23;

FIG. 24E illustrates a perspective view of a discharge collector for receiving the outflow from each of the five selection units of FIG. 23;

FIG. 25 is a side view of three stages of modular sampling units such as another embodiment in a ten long series by six stages;

FIG. 26 is a perspective view of the first stage modular sampling unit of FIG. 25;

FIG. 27 is a perspective view of the modular sampling unit with one sampler shown in axially exploded view with end plates removed;

FIG. 28A is a perspective view of a modular sampling unit illustrating an embodiment of manufacture;

FIG. 28B is an exploded view of the modular sampling unit of FIG. 28A;

FIG. 29A is a perspective view of a modular sampling unit illustrating another embodiment of manufacture;

FIGS. 29B and 29C are end and side views respectively of the modular sampling unit of FIG. 29A;

FIG. 30 are perspective view of three bottom plates spaced for receiving two samplers of the modular sampling unit of FIG. 29A;

FIG. 31A is a perspective view of the inside of one side wall panel of the modular sampling unit of FIG. 29A, the honeycomb structure of the outside shown in hidden lines;

FIG. 31B is a close up view of the groove for receiving the channel's bottom plate taken from area C of FIG. 31A;

FIGS. 32A and 32B are side views of the outside and inside of side wall panels for the modular sampling unit of FIG. 29B;

FIG. 33 is a view of a single stage modular unit according to another embodiment;

FIG. 34A is a perspective view of a last stage of recovery for coupling with a security box, the interface of the recovery tray and box shown in exploded view;

FIG. 34B is a perspective view of the last stage of recovery and security box of FIG. 34A, the interface connected for deposit of precious metals to the security box;

FIG. 35A is a front view of the last stage of recovery and security box of FIG. 34A with interface locking bolts engaged;

FIG. 35B is close up front view of the interface of FIG. 35A, the locking bolts engaged;

FIG. 36 is a top perspective view of the security box illustrating the locking door in the process of closing for removal of the security box;

FIG. 37 is a partial view of the metals entrance to the security box, the recovery tray removed for illustrating the locking bolts;

FIG. 38A is a front, close up view of one side of security box T-slot interface to show one of the locking bolts engaged;

FIG. 38A is a front, close up view of the T-slot interface of FIG. 38B with the locking bolts dis-engaged;

FIG. 39A is a side view of the security box, coupled with the recovery tray during collection; and

FIG. 39B is a side view of the security box, un-locked and un-coupled for automated transport, recovery and cleaning.

### DESCRIPTION

In an embodiment, metals of interest in a slurry ore are concentrated into a recovery stream by sampling, detection and diversion to a recovery or collection stream.

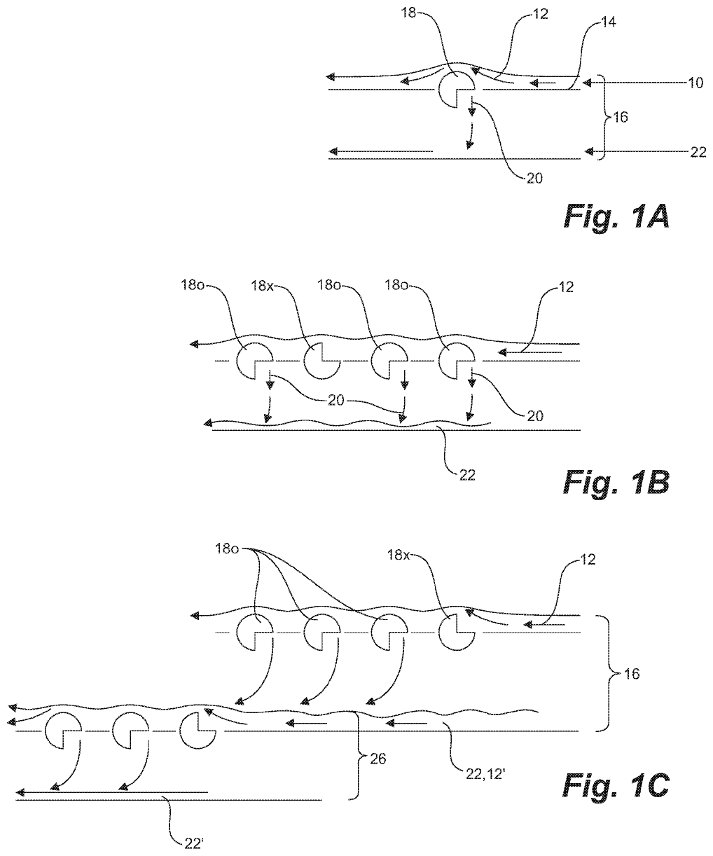

With reference to FIGS. 1A-1C, a feed stream 10 of slurry 12 is delivered to a channel 14. In an embodiment, the ore is pre-processed, mixed with water, and formed into a slurry. The ore can be reduced in size by a variety of known mineral processing crushing and sizing steps. Noble metal-containing rocks or ore, along with dirt and muddy substances are obtained from open (surface) cast-mining, underground cast-mining and panning methods from riverbeds. The ore is crushed, typically corn-sized or a smaller size, and is mixed with water to form a slurry. The slurry is transported in a thin fluid layer through the system for detection and concentration of metals therefrom.

In an embodiment, the crushed ore in this feed stream 10 of slurry 12 is discharged to along a first stage 16.

The first stream 10 of slurry 12 flows along the feed channel 14 to flow over one or more samplers 18. The samplers extend transversely across the feed channel. The shown sampler 18 obtains a sample and, through detection circuitry, analyses the sample for the presence of metals. As shown, if metals are detected, the sampler is actuated to dump or divert a slurry sample 20 to a recovery or second collection stream 22 below the feed channel 14. The samplers 18 can be one or more first stage samplers, 18, 18 . . . with the slurry sample 20 accumulating as a second collection stream 22 being forwarded to a subsequent stage of samplers.

With reference to FIG. 1B, a plurality of first stage samplers 18, 18 . . . can be provided in series, some of which 18o are illustrated as detecting metals in the first stream of slurry and being actuated to dump metal-bearing slurry samples 20 to the second collection stream 22 and, others of the samplers, one shown 18x, that have not detected metals are not actuated.

With reference to FIG. 1C, again, a plurality of samplers 18, 18 . . . can be provided in series. Metal-bearing slurry 20, 20 samples flow to the collection stream 22. The collection stream is directed to a second stage 26 becomes a second stage feed stream 22 to one or more second stage samplers. As is understood, for processes repeated in series, a receiving second stream 22 then becomes a more concentrated yet first feed stream 12′ to a subsequent collection or second stream 22′ and so on. As the context dictates, when the number of stages for a particular embodiment is illustrated, the terms first, second, third are used. In a generic sense, the stages comprise a first stream and a second or subsequent stream.

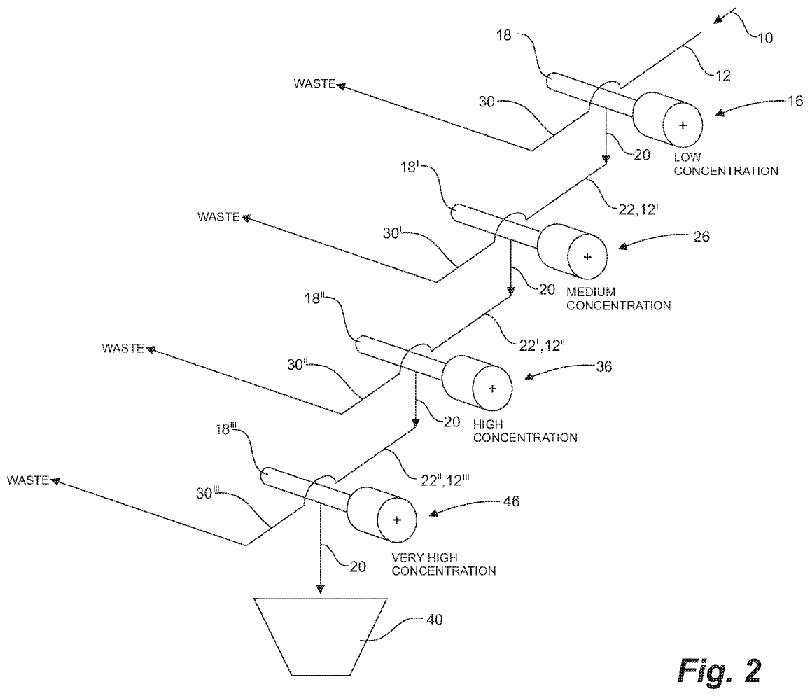

As shown in FIG. 2, the feed stream 10 of slurry 12 can be directed over a first stage sampler 18 for directing metal-bearing slurry to the collection stream. A balance of the first stream of slurry continues to flow as an overflow feed stream of slurry 30 to a subsequent sampler of the one or more samplers. The balance of the feed stream is analyzed in series over additional samplers and any remaining slurry, that is substantially free of metals, is directed to waste. Alternatively, the waste stream may be directed to some final processing stage more suitable to the detection and recovery of trace metals.

The collection stream 22 from the first stage 16 is shown as now forming a second stage feed stream 12′ of metal-bearing slurry. In this second stage 26, the feed stream is further analyzed by the one or more second stage samplers 18′ for extracting metal-bearing slurry from the feed stream 12′ and directing the concentrated metal-bearing slurry to a further collection stream 22′. The balance 30′ of the second stage feed stream, that now contains a minimum amount of metals, is directed to waste.

The second stage collection stream is shown forming a subsequent and third stage 36 feed stream 12″ of metal-bearing slurry. In this third stage 36, the feed stream is further analyzed by the one or more third stage samplers 18″ for extracting metal-bearing slurry 20 from the feed stream 12″ and directing the concentrated metal-bearing slurry to a further collection stream 22″. The balance 30″ of the third stage feed stream, that now contains a minimum of metals, is directed to waste.

Lastly, in this embodiment, the third stage collection stream 22′ is shown forming a fourth stage feed stream 12′″ of metal-bearing slurry. In this fourth stage 46, the feed stream 12′″ is further analyzed by the one or more fourth stage samplers 18′″ for extracting metal-bearing slurry 20 from the feed stream 12′″ and directing the concentrated metal-bearing slurry 20 to the final collection stream 12′″, the final collection stream forming a highly concentrated, metals-rich product 40. The balance 30′″ of the fourth stage feed stream, that has now had the maximum amount of metals removed therefrom, is directed to waste.

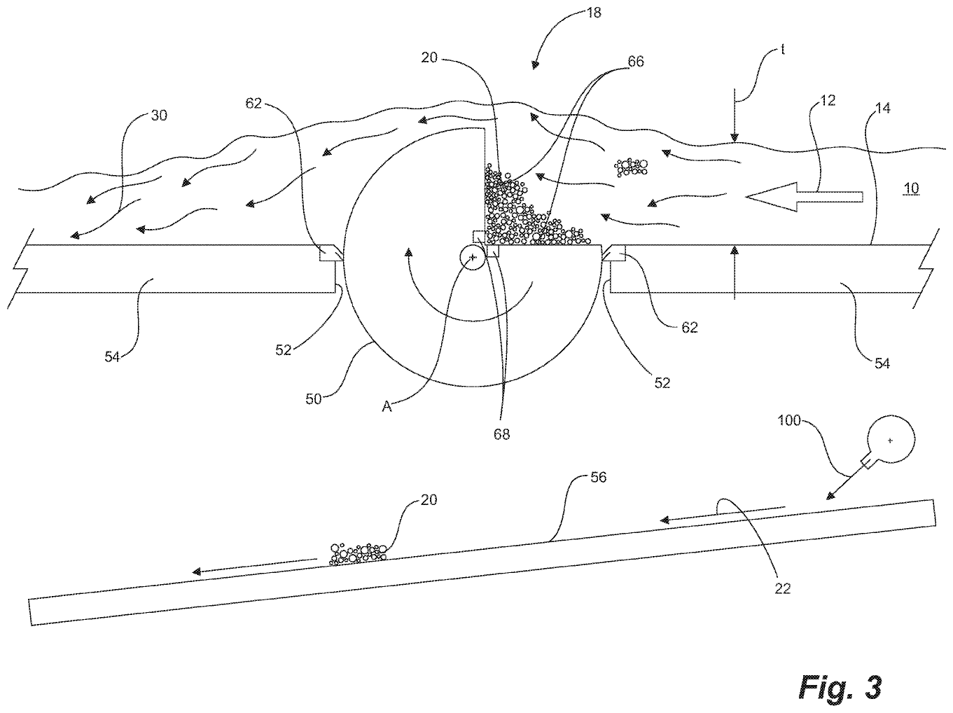

Turning to FIG. 3, in a closer view of the sampler 18 and metals detection, thereby the particular sampler in this embodiment is a rotary sampler 18 comprising a generally cylindrical roller 50 fit to a slot 52 along the bottom of the channel 14. The illustrated sampler and channel 14 are generic to any of the first 16 or subsequent stages 26, 36 . . . although the sampler sizing can vary. The slot 52 in the channel bottom extends generally transverse to the flow of slurry 12. The diameter of the roller 50 is coordinated to be about the width of the slot 52 for substantially filling the slot and forming a generally sealed, contiguous bottom to the channel (by rubber seals or else)

Each roller 50 is inset into the bottom plate 54 surface of the channel 14 and protruding in part above the plate 54 for exposure to the slurry and exposed partially below the plate 54 for access to a recovery tray 56 therebelow. Slurry 10 can flow to, and over, the sampler 18 without significant loss through the bottom 54. Wiper-like, rubber seals 62 or likewise system can be provided to minimize slurry loss between detection cycles. Each roller has a profile or recess 66 extending axially therealong for forming a collection area for the sample 20 of metal-bearing ore. The roller has a sample recess 60 provided therein and is generally aligned with the floor of the channel. A pair 68 of electrodes is located in the profile. The recess 66 is formed along the roller's longitudinal axis A. The recess 66 can be oriented generally into the flow 12 of slurry to maximize sampling of metal-bearing slurry. The recess 66 is circumferentially misaligned above the channel's bottom 54 during sampling so that the full diametric extent portion of the roller seals the slot 52.

The flow rate of slurry 10 is matched to the channel cross-sectional dimensions so as to result in a thin layer “t” of slurry 10 passing over the roller to maximize slurry sampling at the recess.

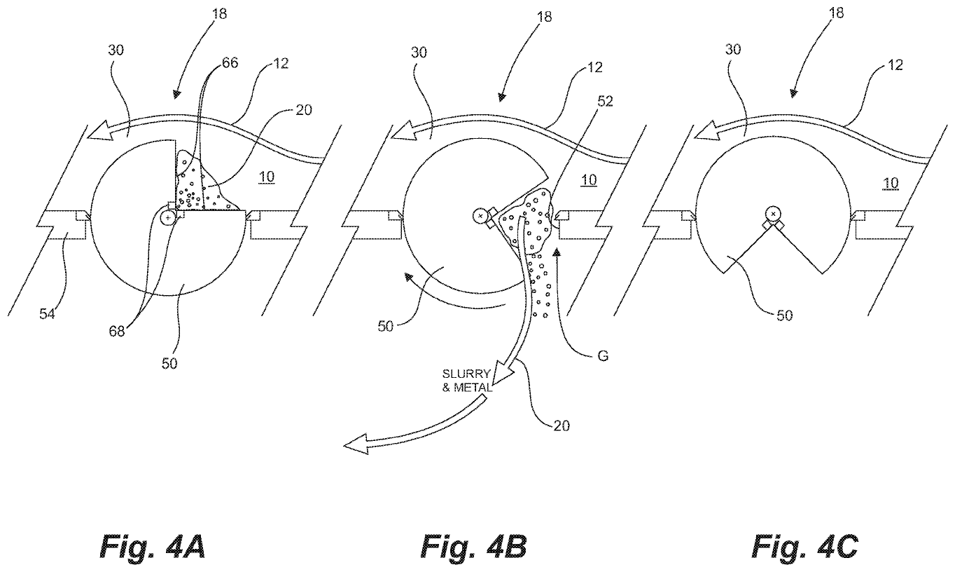

With reference to FIGS. 4A-4C, right to left, the electrodes, adjacent the bottom of the recess 66, detect the presence of metals in the flow of slurry 10. The sampler 18 is controlled to remain stationary until metals are detected (except during an Interval Cleaning Process=ICP). The sampler 18 is maintained in the sampling position FIG. 4A against the flow of slurry thereover. When metals are detected, the sampler 18 is rotated to dump (FIG. 4B, 4C) the sample 20 of detected metals to the collection channel below the sampler roller while the remaining slurry 30 passes above the roller to be collected as waste. When detected, the sampler 18 is actuated to rapidly rotate the recess through the bottom 54 to dump the sampled slurry 20 to the collection stream. As shown in FIG. 4B the recess 66 is momentarily aligned with and opens the slot 52 to dump the slurry sample 20 through a gap G formed in the bottom 54. The process is rapid so as to rotate the recess 66 immediately back into the flow of slurry, in this case the same recess, and re-seal the slot 52.

In an embodiment, the rotation of the sampler rotation is controlled to rotate the recess 66 into and against the stream 12 so as to best retain the slurry sample 20 until rotated for dumping. The gap G between the slot 52 and the bottom 54 of the channel allows the slurry sample 20, containing gold, silver or platinum, to be discharged to the collection stream below. The rotation of the sampler 18 can be un-directional, rotating the recess 66 from the feed stream 12 in a sampling position of FIG. 4A, to a dumping positioned FIG. 4B and continuing through the rotation to position the recess, or another of a plurality of recesses, back into the feed stream (FIG. 4A). Alternatively, the sampler is actuated to rotate the sampler from the sampling position, above the channel bottom, to the dumping position below the bottom, and back again to the sampling position

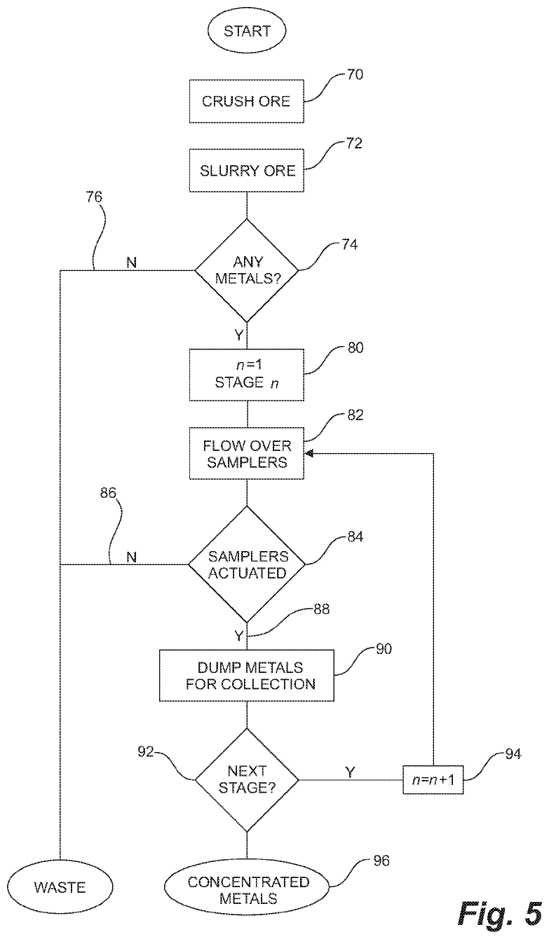

Referring to FIGS. 4A through 4C, and described in the flowchart of FIG. 5, ore is crushed at step 70, formed into a slurry at step 72. A first coarse metal detection can be made on the entire stream. At step 74, if there is no metal detected, the entire stream can be shunted at step 76 to waste, to bypass the samplers. If the first metal detection indicates metals therein, at step 78 the slurry is directed to the first stage (n=1) at 80 to flow over the one or more samplers at step 82.

As shown in FIG. 4C, the slurry 10 fills the recess with a slurry sample 20. If there is no metal detected at step 84 in the sampled slurry 20, the sampler 18 is not actuated at step 84, the flow continuing to the next sampler at step 86 and looping through a detection step 82 and next sampler at step 86 until no more samplers, eventually to waste. In FIG. 4B, if metal is detected at step 84, the sampler roller is actuated 88 to rotate, into the direction of the flow, to retain the metal-bearing slurry sample in the recess, and dump the slurry sample through the slot and to a collection channel at step 90 below the bottom of the feed channel. In FIG. 4C, the sampler roller is rotated, empty, to return to the feed stream in the feed channel to repeat the sampling at step FIG. 4A. If there are additional stages for finer-and-finer detection and collection at step 92 the collection stream forms a subsequent feed stream directed along a subsequent stage's feed channel for processing by a next or subsequent stage (n=n+1) at step 94 of metal detection by one or more samplers. When all stages are complete, the slurry samples have maximum concentration of metals are step 96. Sprays, not shown can wash the recess before the recess is return to the feed stream. The actuation of the sampler is rapid so that the momentary alignment and gap G of the recess and bottom of the channel does not result in much loss of un-sampled slurry.

The sampled slurry 20 that is dumped through the channel bottom 54 forms the collection stream. The collection stream is a flow of slurry 22 that contains a concentrated fraction of metals therein. As shown in FIG. 2, a water stream 100 can dilute the collection stream for purposes of aiding transport, and for ease of handling and sampling at a second stage, if implemented.

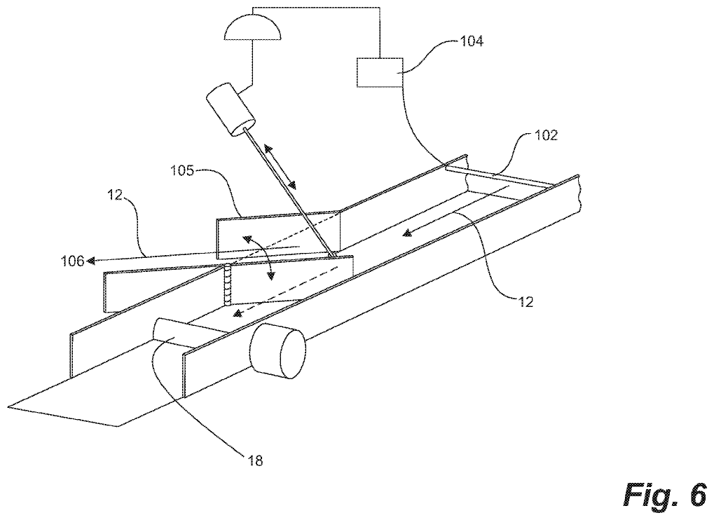

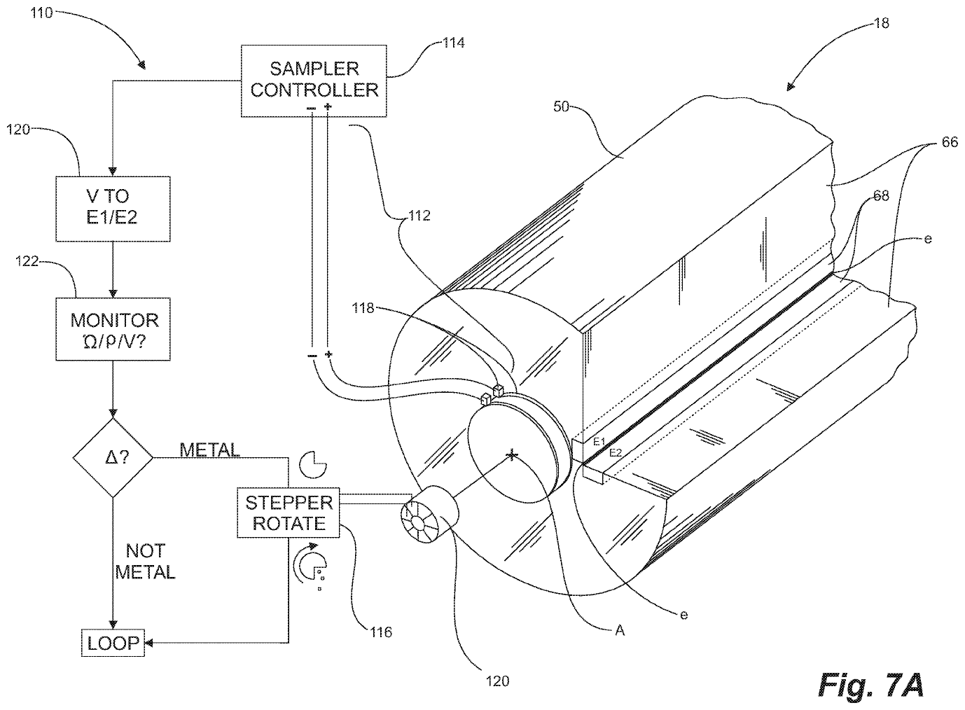

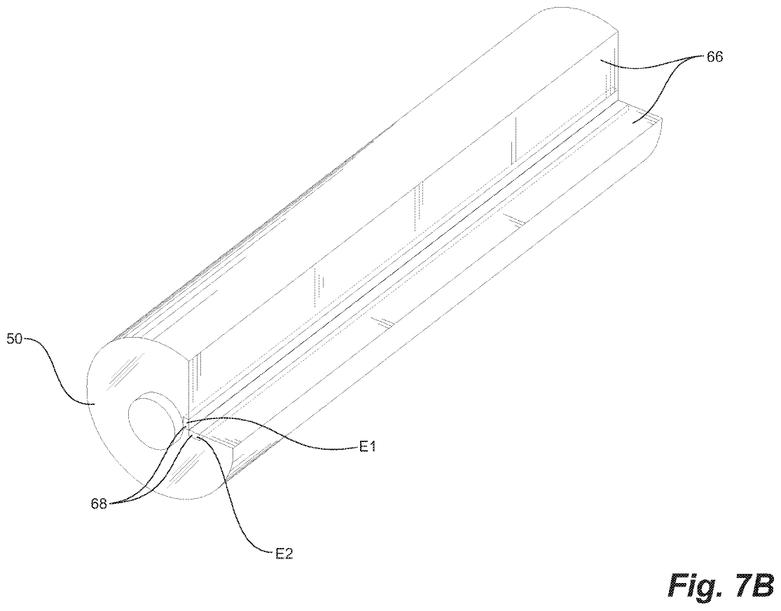

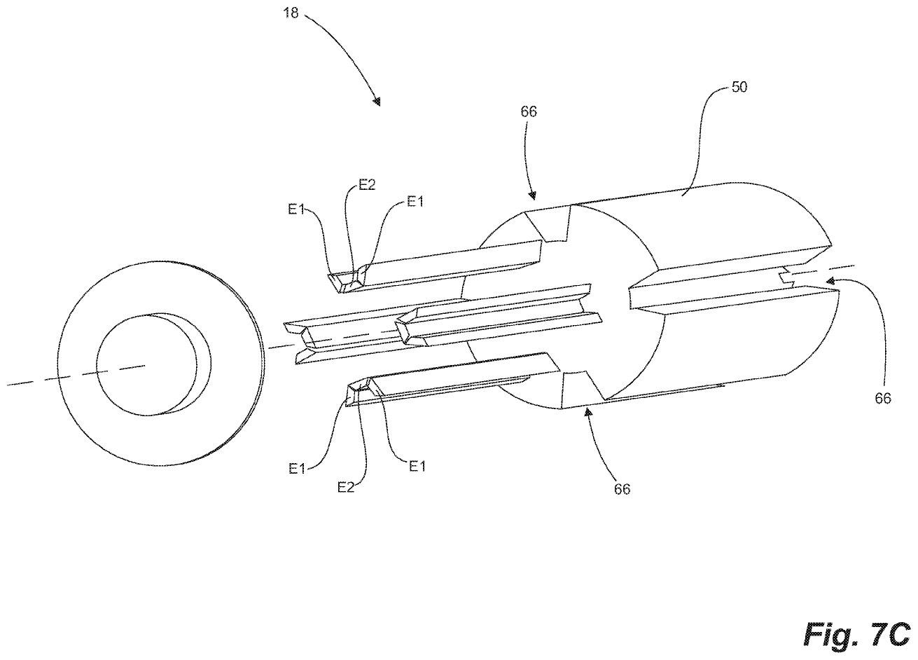

With reference to FIG. 6, in an embodiment of the system, a first metal detector 102 and controller 104 can generally survey the feed stream 12 for metal before passing the feed stream to the samplers 18. An added general pre-detection of metals enables diversion of that portion of feed stream that are absent metals. A side opening gate 105 can be actuated to re-direct the feed stream and minimize needless exposure of the samplers to metal-free slurry. If there are not enough metals therein worth processing, the feed stream 12 is directed along a chute to waste 106 to avoid needless processing by the rotary sampler or samplers. Alternatively, a trapdoor can be operated to divert metal-free slurry to waste. Otherwise, the feed stream 12 is directed over at least one sampler 18. As shown in FIGS. 7A and 7B, metals in the sampled slurry are detected across a pair of electrodes 68 (E1, E2) along the recess. The electrical signal, indicative of metals is conducted from the sampler to circuitry. In the simplest instance, metals are detected as an electrical short across the electrode gap, measured as resistance or capacitance. In other implementations, the amount of metals detected at multiple instances along the electrodes can be distinguished by changes in the signal related to the amount of metals in the sample.

Turning to FIG. 7A, a roller 50 sampler 18 has a pair 68 of electrodes E1, E2, parallel to one another and located at a base of the recess 66. The electrodes E1, E2 are spaced apart and electrically insulated from each other to form a gap e and establishing a normally open circuit. The two electrodes E1, E2 are generally parallel to the roller axis A. The electrodes are electrically connected to a detection circuit 110. The detection circuit comprises an electrical interface 112 with the electrodes, a controller 114, drivers and software for analyzing the electrode output for determining if metals are present. An output circuit 116 provides power and rotation control for actuating the sampler between the sampling and dumping positions.

The controller 114 generally comprises a processing unit, memory or storage, one or more communication interfaces for communicating with other devices via wireless or wired connections, a system bus for connecting various components to the processing unit, and one or more interface controllers controlling the operation of various components. The memory may be RAM, ROM, EEPROM, solid-state memory, hard disks, CD, DVD, flash memory, or the like. Further, the controller typically also comprises one or more displays, such as monitors, LCD displays, LED displays, projectors, and the like, integrated with other components of the controller or physically separate from but functionally coupled thereto. The controller may further comprise input devices such as keyboard, computer mouse, touch sensitive screen, microphone, scanner or the like. Various functions of the controller can be entirely onsite, or offsite. For example, the electrode output can be analyzed onsite for detection and rapid actuation based thereon. Other functions could be performed off site, such as data collection and statistical analysis.

The electrical interface 112 can include slip rings 118 that can be provided to maintain an electrical contact between the movable detection circuit and a non-rotating support structure. Alternatively, to minimize ring and brush noise, wireless transmission devices can be employed.

As shown, the detection circuit 110 can include power 120 and detection circuitry 122. For example, a potential V can be applied across the electrodes E1,E2. The presence of metals at the electrodes can be detected by the circuitry such as through a change in the measured signal across the electrodes. Some parameters that could be employed include a change in resistance Ω, current I or voltage drop ΔV. In an embodiment, the electrodes spaced apart by about 0.2 mm to about 1 mm for processing crushed ore within the slurry of about 0.2 mm to 10 mm with excitation voltages under 1 V. The presence of a signal can be generated by one contact with the electrodes E1,E2 or a plurality of contacts along the electrodes. A variety of electrode arrangements might be employed including longitudinally segmented electrodes for discrete detection or unitary extended electrode for a combined detection signal as shown.

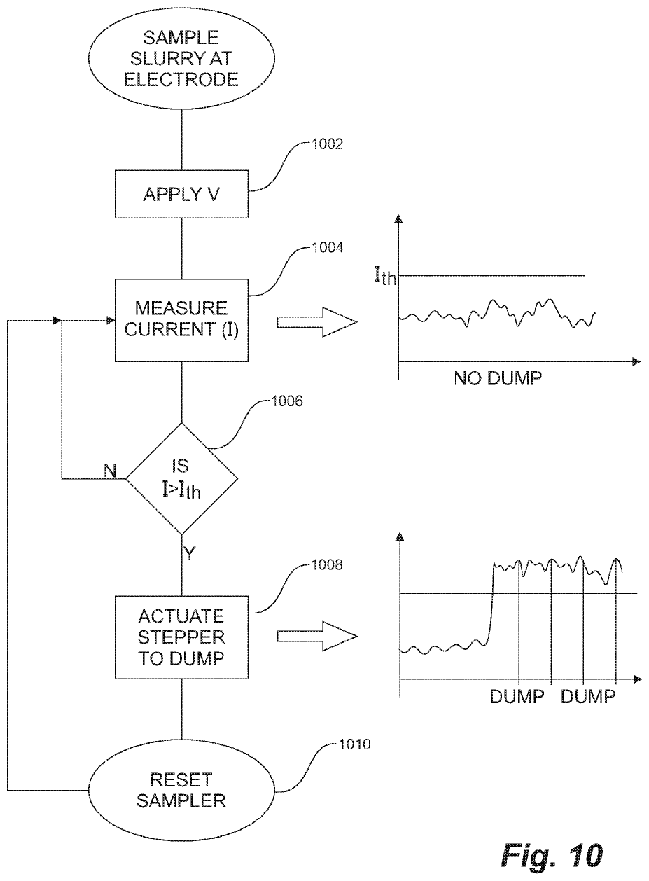

As shown in FIG. 10, in one simple detection embodiment using a change in current I, at block 1002 the electrodes are energized with a potential or voltage V and at block 1004 the current is measured. While many signal processing techniques can be used for noise reduction and pattern matching, for illustrative purposes, a simple threshold technique is shown based on current I. At block 1006, the measured current I is at some background level, not high enough to meet or exceed a threshold Ith then the process loop, awaiting detectable metals. At block 1006, when the measured current I reached the detection threshold Ith, then at block 1008 the controller actuates the stepper motor 120 to index the sampler for dumping and at block 1010, the sampler resets to block 1004 await the next sample containing metals.

As stated, signal processing can be employed to determine if the measured change exhibits a pre-determined behavior or exceeds a threshold. Signals are indicative of the presence of metals, such as those exhibiting a signature or a magnitude above a background, or threshold. Calibration techniques, for a sampler 18, or a sampler's electrodes E1,E2 can be used either for establishing signatures or thresholds indicative of metals or for determining background or noise. If metals are detected, then the detection circuit actuates the sampler to dump a slurry sample. In the case of the rotary sampler, an actuator, such as a stepper motor can be actuated to rotate the sampler as described above in FIGS. 4A-4C. The process repeats as the feed stream 12 continues over successive samplers.

The controller 114 includes a Stepper Control unit (SCU) that can steer the rollers, namely for orienting them to receive slurry in the recess for sampling, to dump the recess and for clearing jams. The SCU controls the stepper motor 120 to orient the rollers to a pole-position to receive slurry. If there are four circumferentially spaced recesses 66 at 90 degrees (FIG. 7C), then there are four pole-positions. The SCU ensures a recess 66 is oriented for sampling. An LCD-Display can show error codes and the status of the stepper motor 120. A LED can indicate the location of problems with the sampler. The SCU also has also an interface, such as buttons, for configuration and reset. The SCU will detect a jam upon dumping the recess and enable reversal to clear the jam. Alternatively, or in addition, the SCU can periodically reverse the roller direction off of the pole-position to clean the sampling recess.

Protection of the stepper motor 120 and sampler 18 is provided with a clutch 122 between the stepper motor 120 and the roller 50. Further, the electrical detector E1, E2, which rotates on a roller-type of sampler, is electrically connected through a rotary connection.

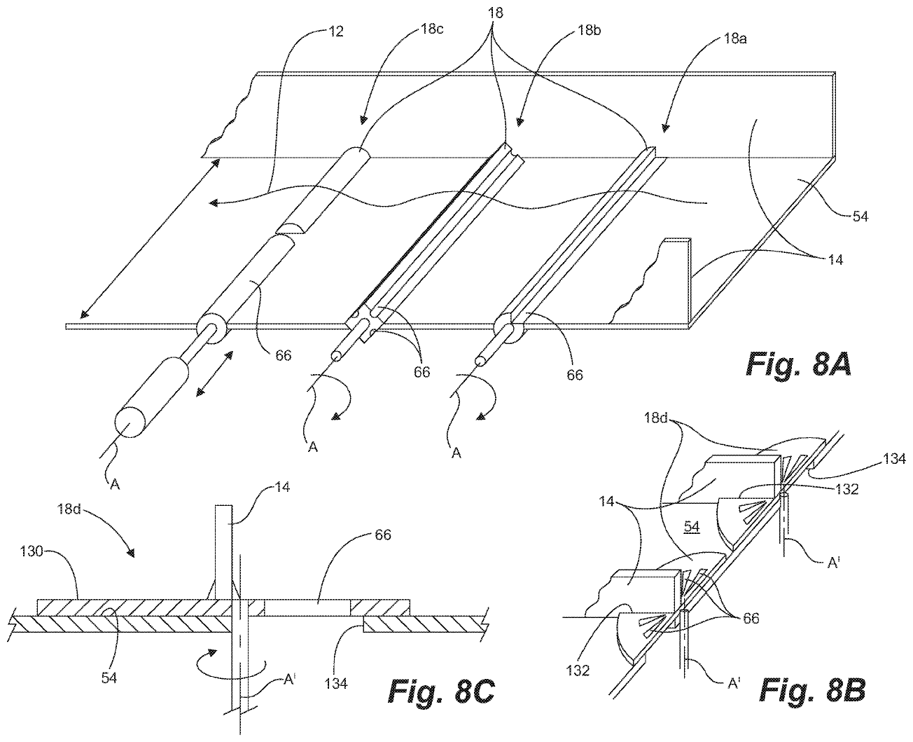

With reference to FIGS. 8A through 8C, alternate samplers 18a, 18b, 18c, 18d contemplated include rotary samplers—having rotary axes along either horizontal or vertical axes. As shown in FIG. 8A, a cylindrical sampler 18a having a circular roller profile, of FIG. 7A, is shown with the axis A extending transverse to the feed stream 12. Similarly, a rotary sampler 18b having a lobed, triangular or polygonal cross-section or profile is shown. The illustrated rotary samplers extend transverse to the feed stream 12 and have a horizontal axis A, with the slurry flowing over a longitudinal extent of the sampler 18 exposed to the slurry in the channel 14. In another example of a transversely-extending sampler 18c, one or more linearly-actuated samplers might be actuated from the side of the channel to extend transversely into and out of the feed stream. In FIGS. 8B and 8C, a rotary table sampler 18d can have a vertical axis A′, with a table 130 extending from the side of the channel 14 into the feed stream and rotatable through a slot 132 in the side wall or side of the channel 14 for dumping the collected slurry sample from recess 66 to the collection stream below. As shown in FIG. 8C, the table 130 can rotate above a continuous channel bottom 54 to collect the slurry sample in the recess 66 in the table 130. Upon detection of metals using detectors in the recess 66, the table 130 can be rotated to align the recess 66 with a port 134 outside the feed channel 14 so as to fall from the recess 66 to dump into the collection stream there below.

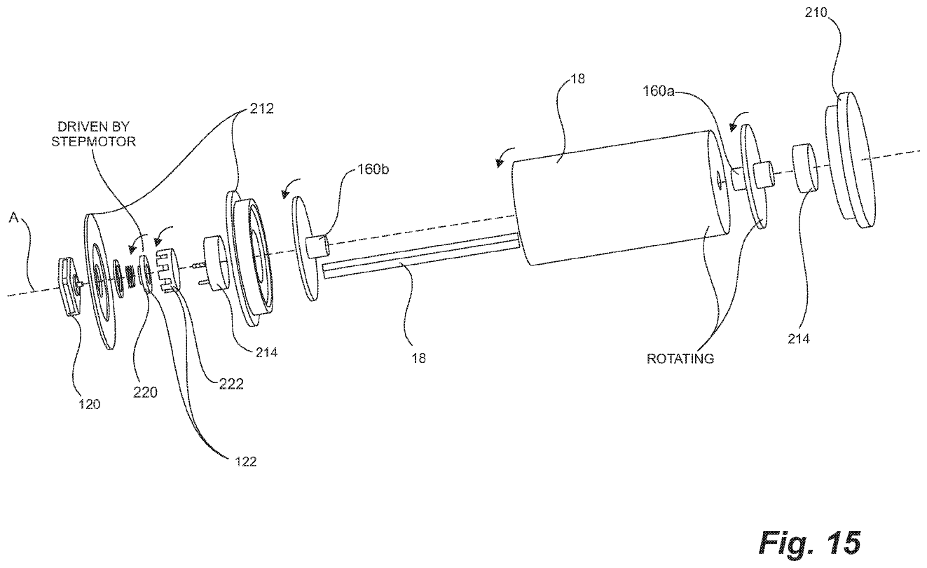

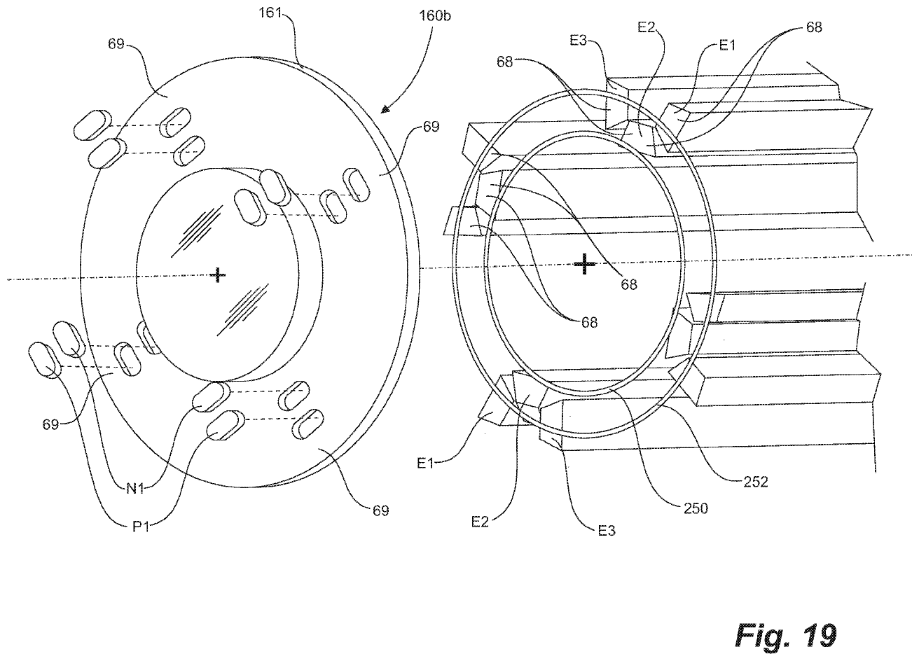

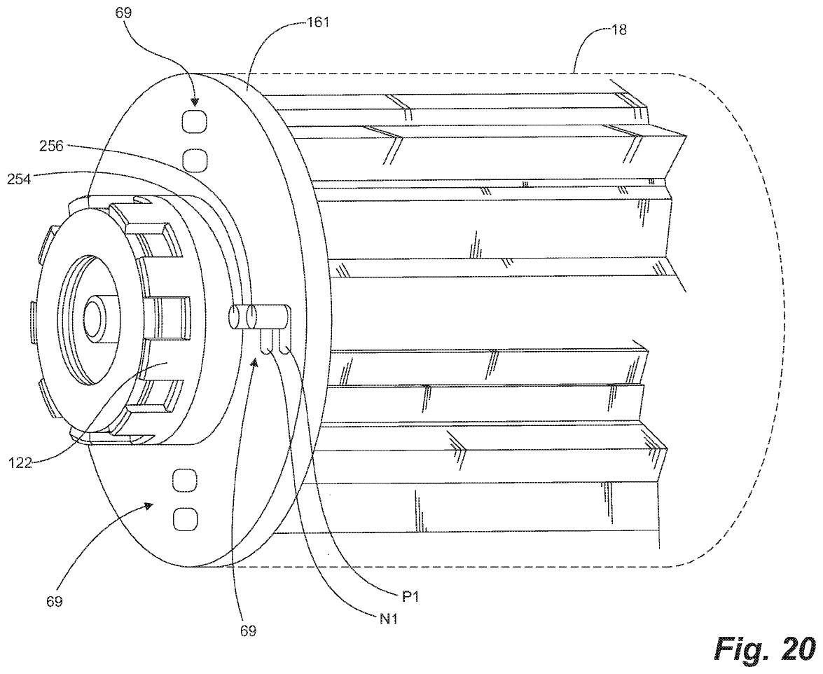

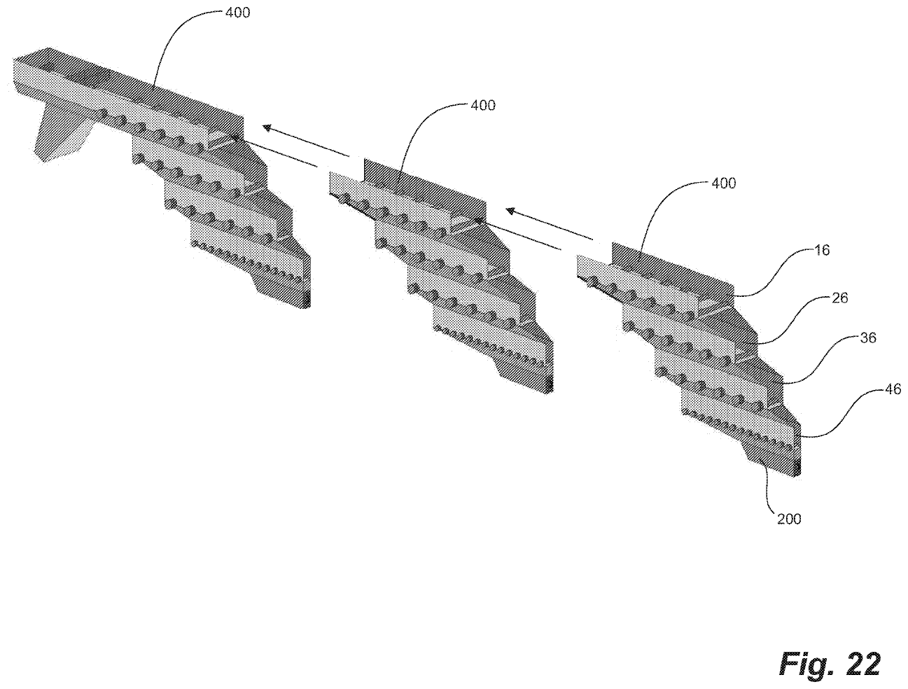

With reference to FIGS. 15 through 20B, a roller-type of sampler 18 is illustrated with its component parts for rotary driving and support. In FIG. 15, the components are shown in exploded representation along axis A and in FIG. 22A, the components are shown assembled.

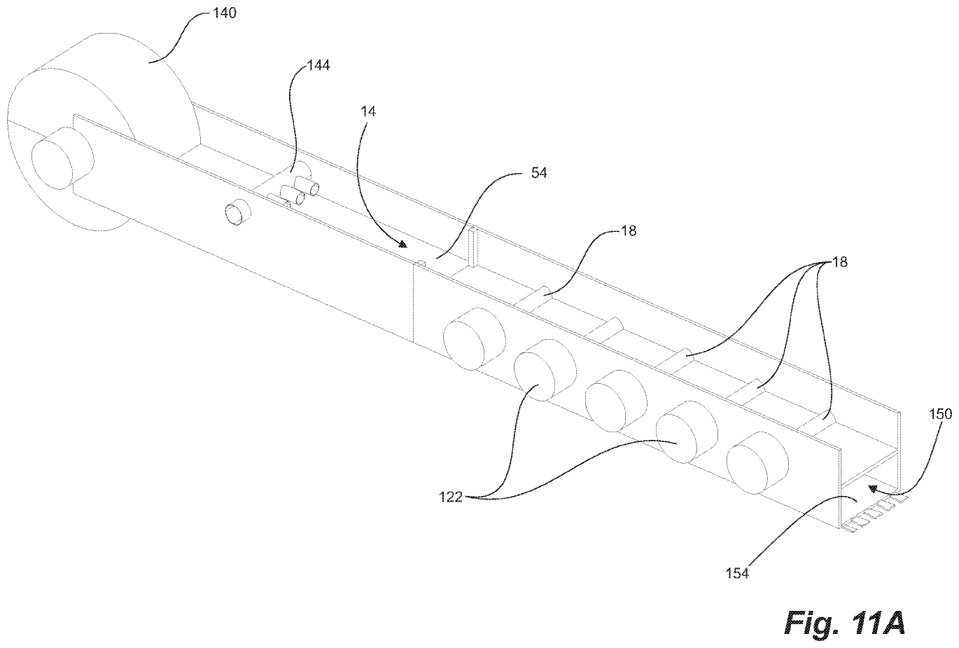

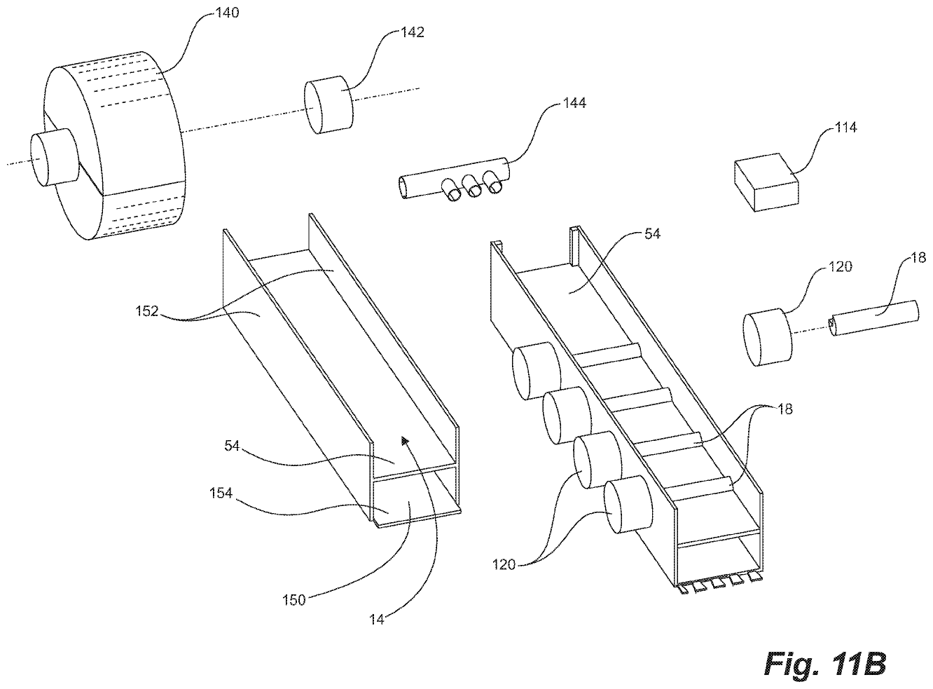

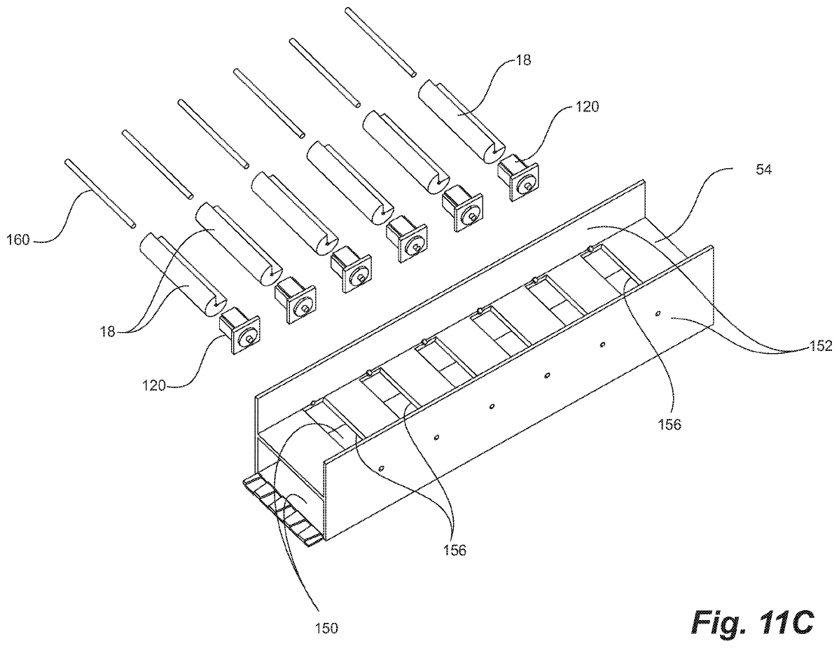

The sampler 18 is supported in the apparatus of a single stage system of FIG. 11C, such to side walls 152 of channel 14. One passive end of each roller is supported by an end plate 210 and an active end of the roller is supported by an end plate 212. The ends plates 201,212 are secured to the side walls. The roller 50 is fit with an axle shaft 160 extending therethrough along A, or supported otherwise, with shafts 160a, 160b at opposing ends of the roller. Shafts 160a,160b are rotatably supported at the end plates through bearings 214, 214.

The stepper motor 122 is secured to the end plate 212 and the shaft extends therethrough, along axis A, to rotationally drive a first driving clutch portion 220. A second driven clutch portion 222 is secured to the roller 50 through shaft 160b. If the roller 50 stops rotating, due to a jam or resistive torque above a threshold, the driven clutch portion 222 stops rotating, and driving clutch portion 220 can continue to rotate relative thereto, the stepper motor 120 detecting an operational change at the roller. Driving and driven clutch portions are rotationally coupled until a threshold torque is exceeded at which time the components slip.

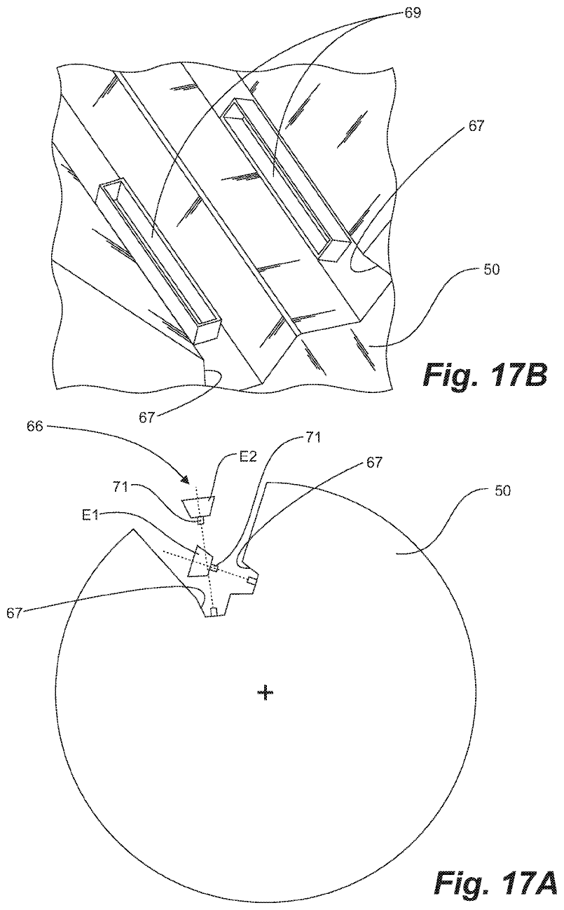

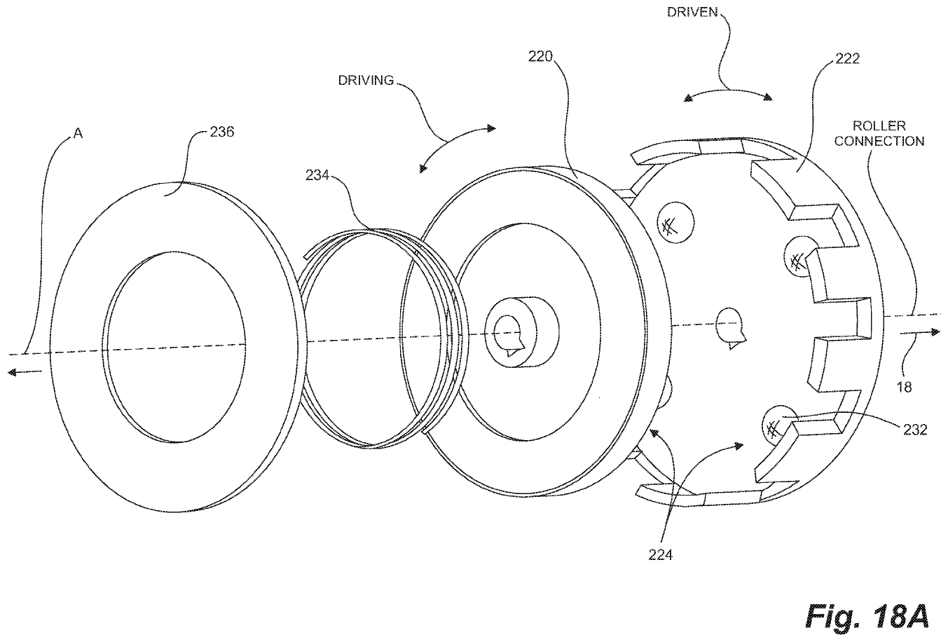

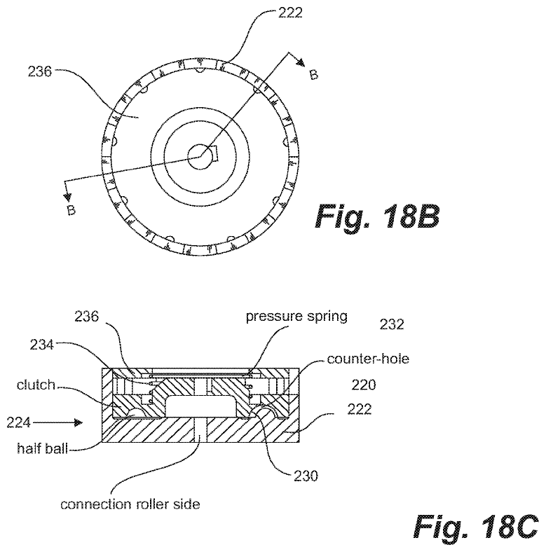

Turning to FIGS. 17-18B, the clutch 122 comprises the driven clutch portion, affixed for co-rotation with the roller, and the driving clutch portion affixed rotationally for co-rotation with the shaft of the stepper motor 120. Both portions 220,222 are plates having a driving interface therebetween 224. The driving interface 224 can comprise complementary ramped recesses 230 and protrusions 232, rotation of protrusions relative to the recesses acting to drive the protrusions axially from the recesses. The driving clutch portion is axially biased to forcibly retain the protrusions axially in the recesses for co-rotation of the driving and driven portions. As the resistive torque of the driven clutch portion increases, the driving portion 220 the protrusions 232 ramps axially out of the recesses 230 until clear and the clutch disengages. As shown, the protrusions 232 are generally hemispherical and the recesses 230 are complementary hemispherical indentations. Other angled or ramped interfaces are also suitable.

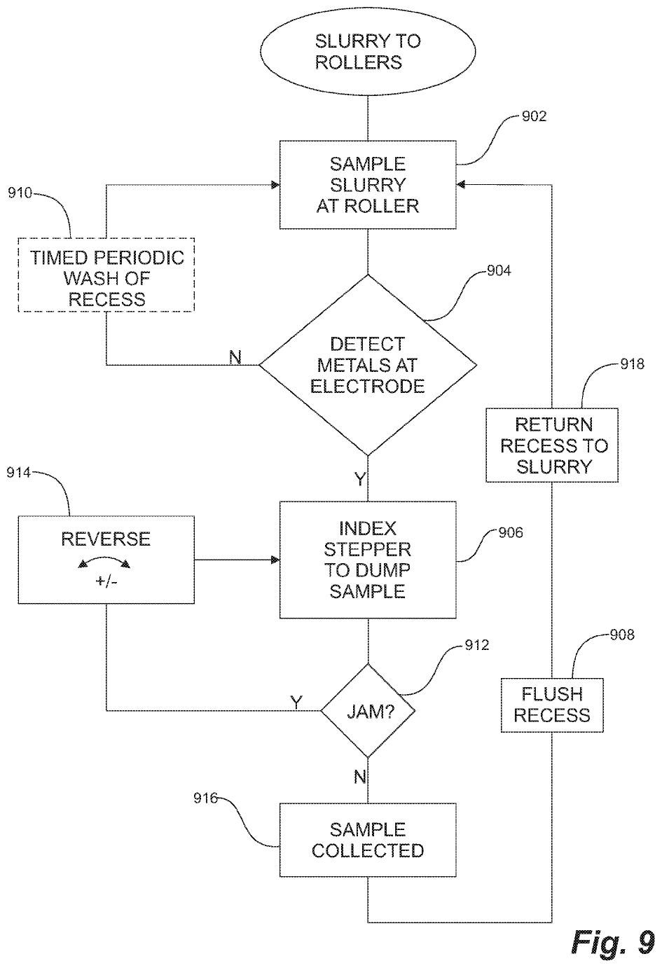

The driving portion 220 is biased by spring 234 sandwiched between a pressure plate 236 that can be secure to the driven portion 222 and the driving portion 220. The driven portion 222 can be fit with a speed sensor for aiding in remote detection of roller rotation anomalies. The driven portion can be castellated such for fitting of Hall Effect sensors, for weight reduction, access or debris relief. With reference to FIG. 9, the process of metal detection can include several maintenance functions including washing the recess 66 and clearing a jammed sampler. For a roller 50 type of sampler 18, an Interval Cleaning Process (ICP) can periodically or frequently turn the roller 50 backward frequently to clean the recess 66. The recess 66 can be oriented to wash an old slurry sample 20 out before reorienting the recess for fresh sample. Alternatively, the roller 50 can be rotated entirely over to permit dumping of any old sample out of the recess by gravity. The SCU rotates the roller in reverse or forwards to the select the next pole-position with the recess 66 facing the slurry feed stream 12.

A wash step, or periodic wash step can be employed to ensure the recesses and electrodes are operating at optimal detection efficiency. Further, as each sampler is directing a slurry sample from a first environment or feed stream 12, to a second environment or collection stream 22, there is a possibility of a periodic jam intermediate sampling and dumping positions. Accordingly, and applicable to the above additional process embodiments in the context of a rotary-type sampler 18, at Block 902 slurry is sampled at the roller sampler. If metals are detected at Block 904, the roller is indexed, such as by stepper motor at Block 906, to dump the slurry sample. As the recess is basically empty, it is also an opportune time to flush the recess at Block 908 and condition the electrodes for optimal detection. Flush sprays can be arranged below the bottom of the channel and directed along the recess during dumping, for mixing and addition to the collected stream, or thereafter. If no metals are detected at Block 904, the sampling continues. As there could be a period of time that slurry sample remains in the recess, an optional periodic flush can be applied at Block 910 to empty the recess of stagnant sample and enable collection of a fresh slurry sample.

If, during indexing, the roller is jammed at Block 912, then the controller can quickly sense interruption of the rotation of the roller, reverse and advance at Block 914 until the slurry sample 20 is successfully dumped. As is the usual case, if there is no jam at Block 912, the slurry sample is collected at Block 916, the recess can be flushed at Block 908 and the controller returns the recess, of a recess of multiple recesses, into the slurry stream 12 at Block 918.

Turning to FIG. 11A, for a description of the system overall and the relationship of the components, a single stage system is illustrated. For improving the efficiency of collection, one or more samplers 18, 18 . . . are provided and preferably an array of samplers is provided in series for collection efficiency. The plurality of samplers 18, 18 . . . are mounted in series across a chute or feed channel 14, arranged transverse to the channel 14, and having a portion exposed to the flow of slurry. Rotary or roller samplers 18 are shown, having an upper portion exposed to the slurry. The feed channel 14 has a first bottom 54 for directing the slurry to and over the samplers 18. Slurry flows along a floor or upper surface of the bottom 54.

The system can include a pre-sampling feed assessment or conditioning apparatus. The slurry can be pre-conditioned or assessed for suitability, either to the presence of metals or to a gradation of the particles within the slurry. Feed conditioning can include screening or other sizing steps and removal of oversize including foreign materials. Assessment can include a determination if the slurry contains metals of interest or not. If not, then there is no need to perform the sampling step and the non-metal bearing slurry can be directed to waste.

Optionally, in advance for either mixing the slurry or for size management, the ore is processed through a trammel 140. The trommel can include a magnet for removal of scrap metal and is sized for removing oversize from the bore for directing to waste or resizing.

In one embodiment trommel 140 screens the slurry for either oversize solids or for unacceptable metals, such tramp metal from the mine. The trommel 140 can include a trommel drive 142. A water addition header 144 is provided for aiding with slurry formation or transport.

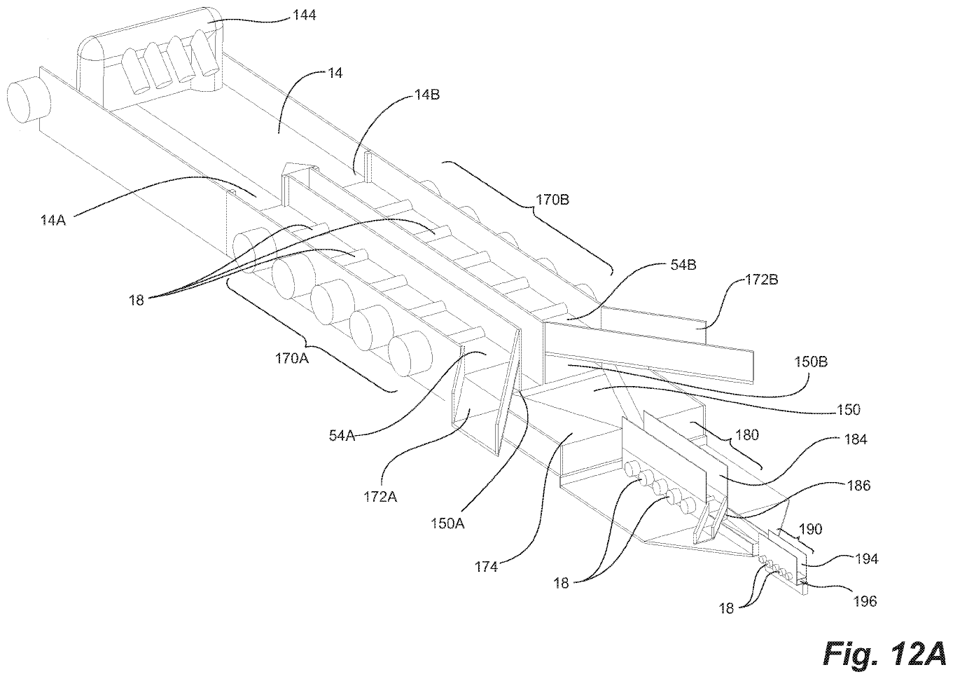

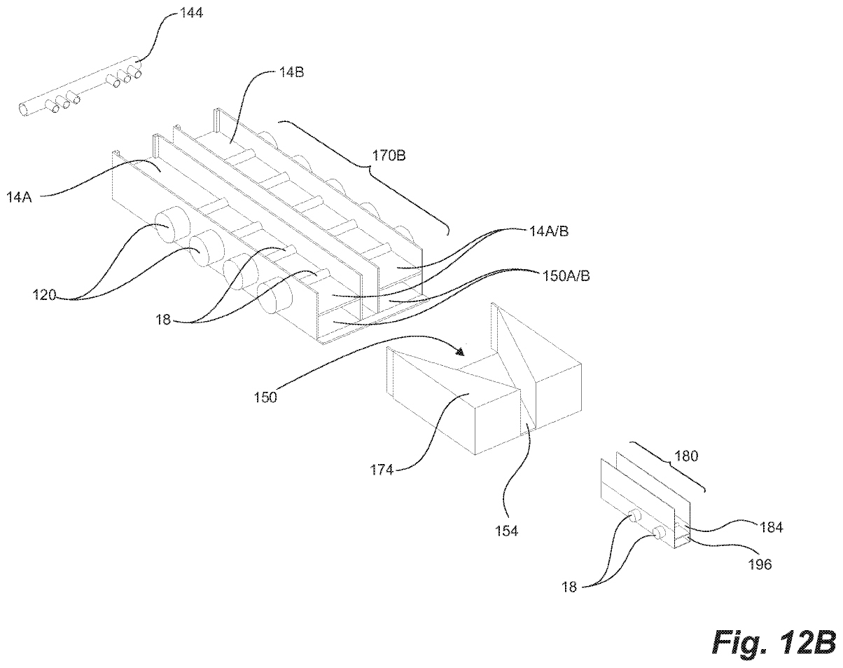

Alternatively, before introducing water for forming the slurry, crushed ore is discharged from the trommel 140 and thereafter combined with water in the feed channel 14 to form the feed stream 12 of slurry. As shown in FIG. 12A, a water header 144 can be provided for the introduction of water such as through one or more sprays directed across the transverse width of the channel.

As described above, when metals are detected at one or more of the samplers 112, a slurry sample from the respective sampler is diverted for collection. Controller 114 actuates a stepper motor 120 or other actuator, for the respective sampler 18, moves the slurry sample in the recess containing metals, from the feed chute 14, through the feed channel's bottom 54 to the collection channel 154. The collection channel 150 is shown located beneath the feed channel 14 and comprises a second bottom 154 for directing the concentrated collected slurry 22 to a subsequent stage of recovery.

As shown in FIG. 11B, the feed and collection channels 14, 150 are typically fabricated as a two side walls 152 having a first plate forming first bottom 154 and a second plate forming the second bottom 150.

Turning to FIG. 11C, the first bottom 54 of the channel 14 is fit with a plurality of transverse slots 156 sized to receive the samplers 18. The samplers 18 fit the slots 156 to form a substantially continuous feed channel 14 for directing flow of slurry from sampler 18 to sampler 18. The rotary samplers depicted are shown with shafts 160 rotatable fit to bearings in the side walls 152 of the first channel 14. As shown, each of the plurality of samplers 18 and corresponding slots 156 are like-sized, however it is contemplated that each sampler 18 and slot 156 in series could be of diminishing and corresponding size, increasing size or of variable sizes. Further, the configuration of recesses 66 in samplers 18 can vary.

Further, for increasing the rate of processing, one can provide two or more feed streams 12 in parallel. The need for parallel streams is most apparent at the first stage of processing where the largest flow of feed slurry is processed for coarse detection of metals. Each subsequent stage has a reduced flow, being a more concentrated collection stream, and thus the number of parallel feed streams can be reduced in number, perhaps down to one channel.

As discussed, the system can include multiple stages and parallel streams. The components of parallel streams are numbered with the same numerical reference values, but with added letters A,B,C, for the same component, only located on the parallel unit. For example, a single stream system has one channel 14. A system having two streams in parallel has two channels 14, numbered channels 14A and 14B. A system having five parallel streams has five channels 14A through 14E.

Turning to FIGS. 12A and 12B, equipment is shown implementing parallel first feed channels 14A,14B. Slurry is introduced and water added through a common header 144 for simple hydraulic division of the slurry into two parallel channels 14A,14B. Two series 170A,170B of samplers 112 are installed along the respective channels 14A,14B. Un-marketable slurry that flows over the series of samplers 18, without being diverted, is directed to waste through end chutes 172A, 172B. Beneath channels 14A,14B, are collection channels 150A,150B, although both channels 14A, 14B could dump samples to a common second channel 150.

At a discharge of the second channel 154 or channels 154A,154B, a funnel 174 directs the collection streams of sampled slurry to a second stage of selection having its own series 180 of samplers 18, 18 . . . .

The second stage comprises a second stage feed channel 184, its own series of samplers 18, and a collection channel 186. The first stage collection channel 150 is fluidly contiguous with, and feeds its slurry into, the second stage feed channel 184. As the flow rate of concentrated slurry is significantly reduced, the second stage could comprise a single stream, and further, the samplers 18 can be smaller or have different arrangements of recesses for detection of metals in the diverted subset of slurry concentrate. At the end of the second channel 150, the second collected feed stream is directed through chute 184.

At a discharge of collection channel 186, the slurry is routed to a third stage of selection having its own series 190 of samplers 18, 18 . . . . The third stage comprises its own feed channel 194, the series of samplers 18, and a collection channel 196. The second stage collection channel 186 is fluidly contiguous with and feeds its slurry into the third stage feed channel 194. Again, as the flow rate of slurry is significantly reduced, the third stage comprises a single stream and smaller samplers.

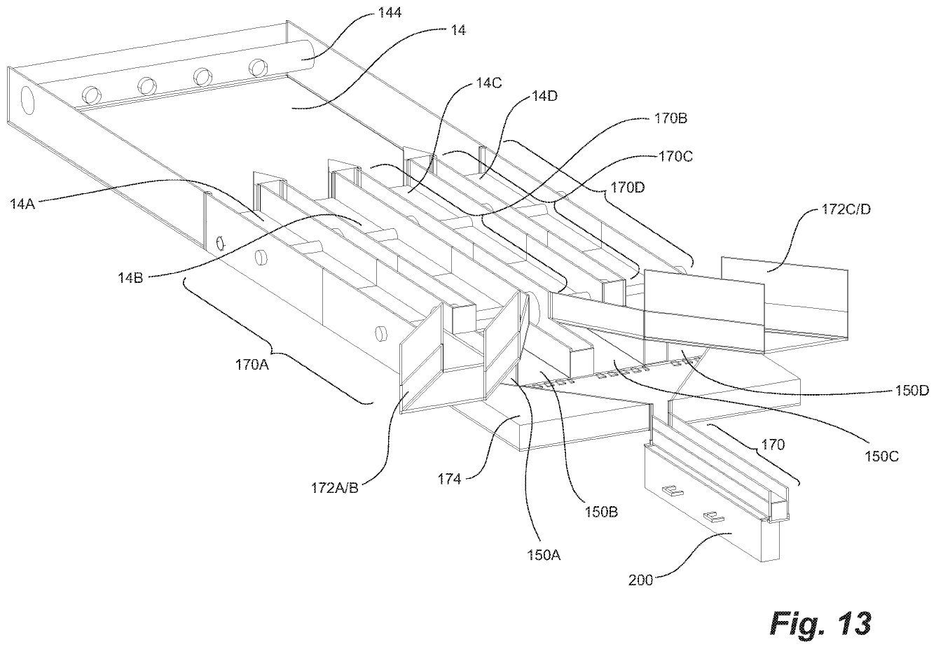

Turning to FIG. 13, a two stage, four parallel feed stream apparatus is shown. The equipment is shown implementing four parallel first feed channels 14A,14B,14C,14D. Water can be added to the slurry in the channel or channels through a common header 144 for simple hydraulic division of the slurry from a main channel 14 into the four parallel channels. Four series 170A,170B,170C,170D of samplers 112 are installed along the respective channels 14A,14B,14C,14D. Each series of samplers 18,18 . . . is equipped with its own actuators 120, 120 . . . . Again, un-marketable slurry, absent useful levels of metals, is directed to waste through end chutes 172A/B,172C/D. Two chutes are shown, each incorporating two adjacent feed streams. Beneath the four channels are one to four collection channels, numbered uniquely as 150A,150B,150C,150D.

At a discharge of the second channel or channels 150A-150D, funnel 174 directs the collection streams of sampled slurry to a second stage of selection having its own series 170 of samplers. In this embodiment, the second stage is the final stage, the collection stream being deposited into a recovery tray 200.

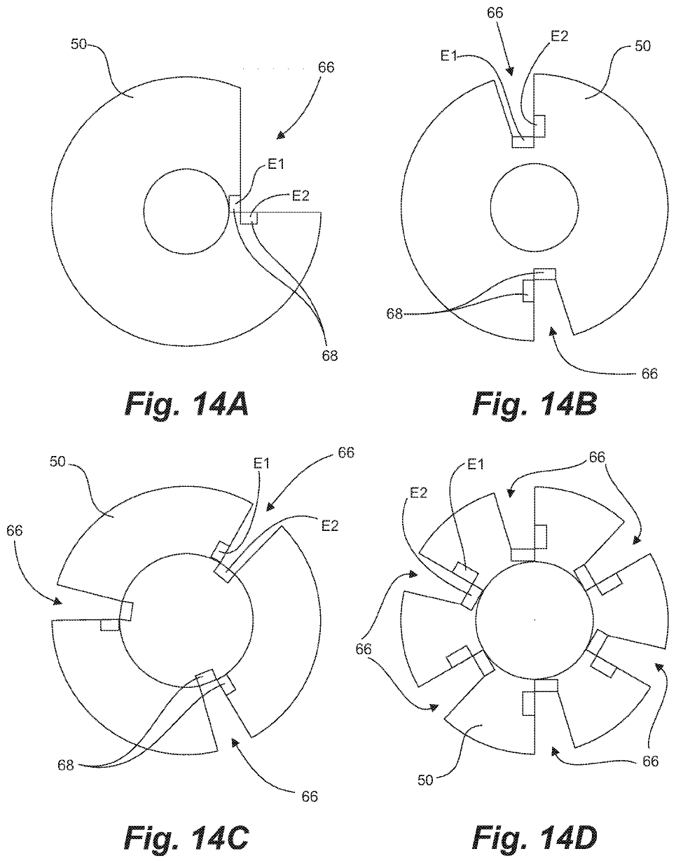

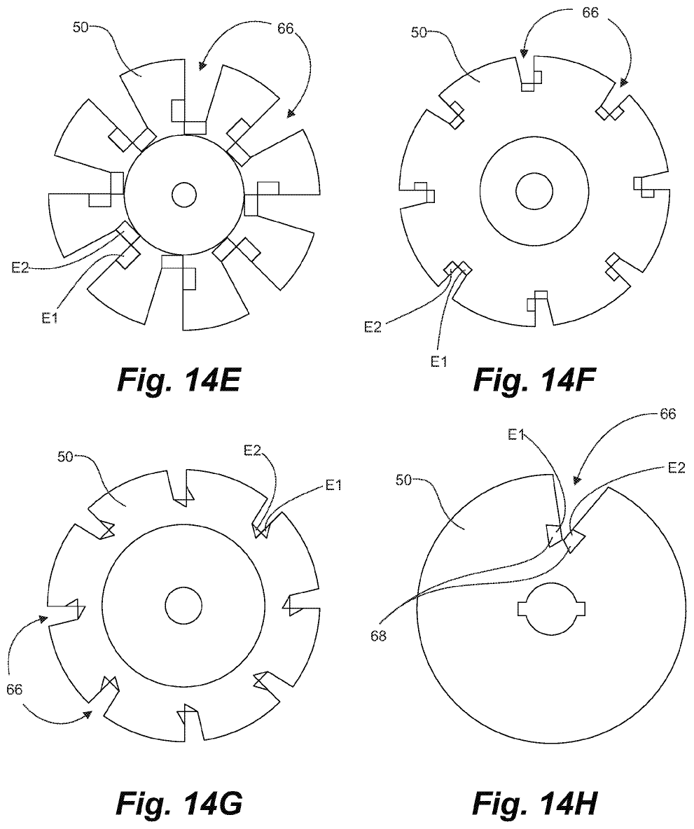

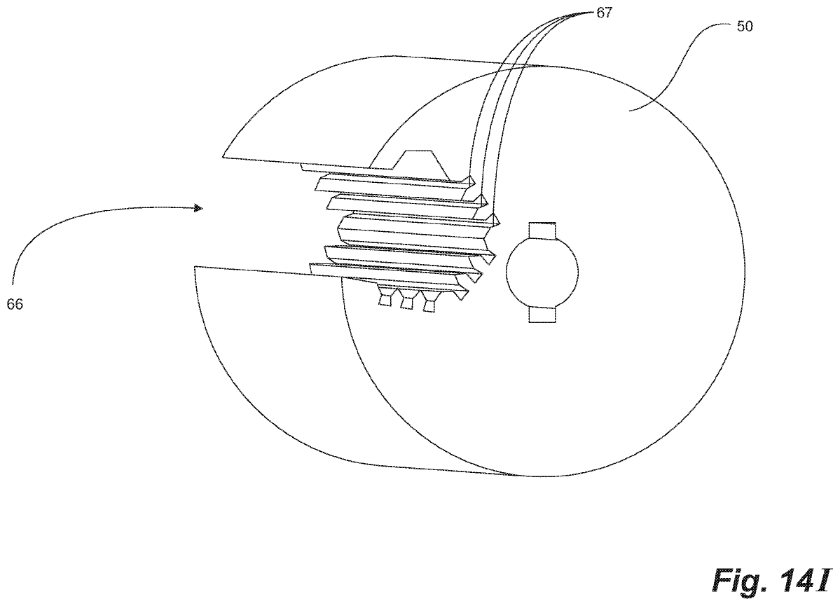

With reference to roller-type samplers of FIGS. 14A to 14G, sampler recesses 66 can be configured in a variety of forms and variety of electrodes. The number of recesses 66 can be one or more, the number being based in part on the physical arrangement and capacity about the sampler circumference and the size of crushed ore particles in the slurry.

As shown in FIGS. 14A through 14E, cylindrical samplers 18 or rollers 50 can be fit with one large recess 66 for initial detection and sampling, and as the slurry flows along a series of samplers, or from stage to stage, or both, the recesses could become progressively smaller and larger in number per sampler. FIGS. 14A through 14E illustrate one, two, three, six and eight recesses 66 respectively. In the case of multiple recesses 66 and electrodes E1,E2, each recess is spaced equally about the circumference. Each recess 66 has one or more pairs of electrodes, typically arranged about the bottom of the recess towards the axis. Electrically, electrodes are provided in pairs 68. Typically, each electrode pair 68 comprises two electrodes E1,E2, and two pairs 68,68 can be provided as three electrodes E1,E2,E3, having a common ground E2 and so on.

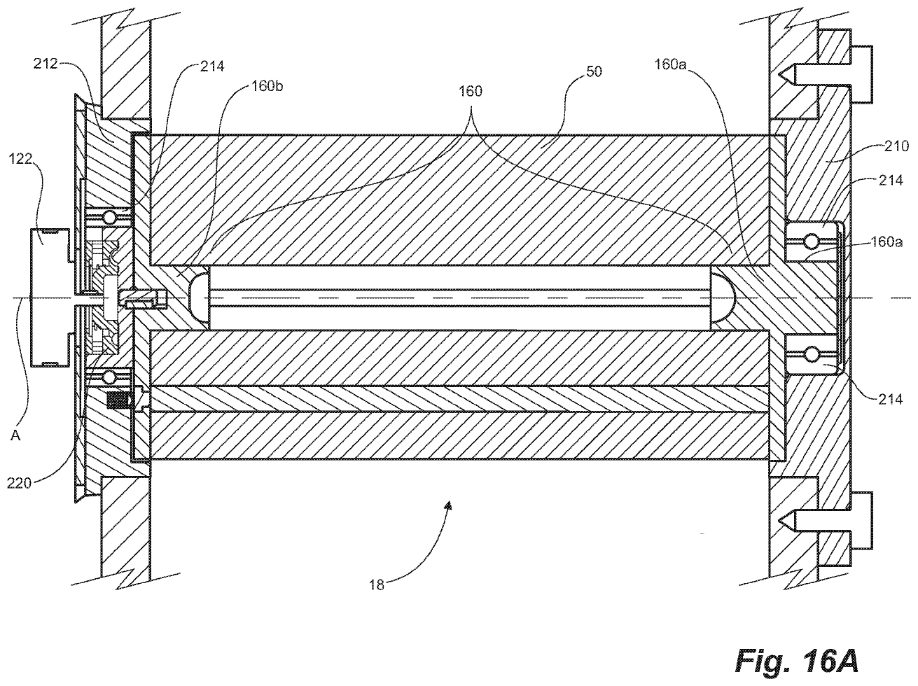

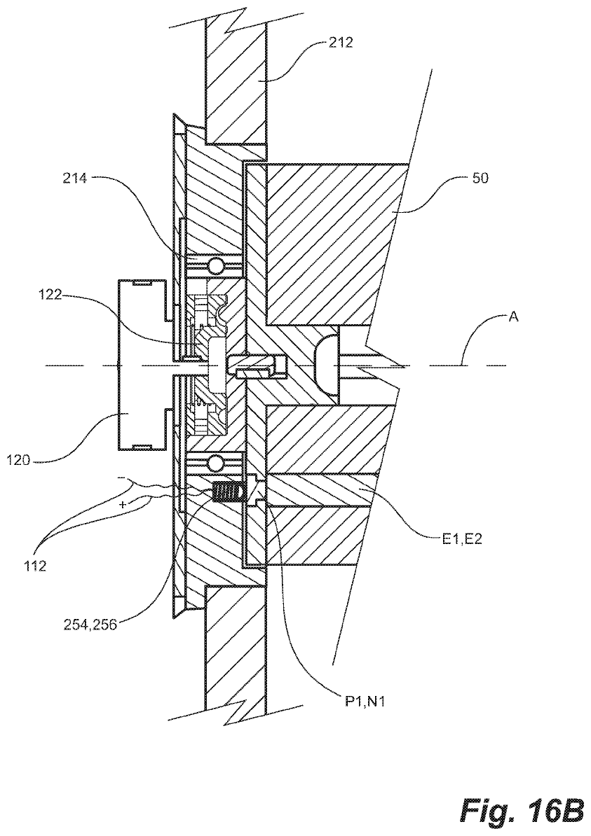

As shown in FIGS. 19, 20 and 16B, the electrodes 68 are connected through a rotary electrical connection system, to interface between the rotating roller 50 and the stationary end plate 212. Four recesses, not shown, support four sets of electrodes actually comprise eight pairs of electrodes 68. The electrodes are shown in floating isolation as they would appear when fit to a supporting roller. Each of the four sets of electrodes comprises three electrodes E1,E2,E3, one ground and two positives, or vice versa, providing two detection gaps in a single recess. Two circular electrical contacts 250, 252 are provided, one circular contact 250 for ground and one circular contact 252 for a positive terminal. The ground electrodes E2 for each set are electrically connected to the ground circular contact 250 and the positive electrodes E1,E3 for each set are electrically connected to the positive circular contact 253.

With reference to FIGS. 17A and 17B, electrodes can be readily installed and replaced using an electrical pin and socket arrangement. Electrodes can be replaced if worn or damaged. As shown, an electrode E1 or E2 can have one of more pin connections 71 for removable electrical connection with a corresponding one or more sockets 69 along a supporting electrode slot 67.

The roller shaft 160b is fit with a plate 161 for both affixing the shaft 160b to the roller and for supporting one or more pairs 69 of electrical point contacts N1,P1 are aligned radially with their respective the circular contacts 250,252. For purposes of rotational alignment and location of the recesses 68, one pair 69 of the point contacts N1,P1 is provided at, or otherwise indexed angularly with respect to each recess. One pair of brushes or spring contacts 254,256 are provided and supported by the non-rotating end plate 220 as shown in FIG. 16B. The spring contacts are connected to the electrical interface 112.

Four pairs 69 of point contacts N1,P1 are aligned rotationally with four recesses or pole position in electrical terms. When the spring contacts 254,256 are in electrical contact with a pair 69 of point contacts N1,P1, the controller is aware of the alignment of the recess to the slurry stream for sampling and detection of metals of interest is relevant. When misaligned, the recess is not in the sampling position and instead is in the process of dumping a collected sample.

In FIGS. 14E to 14G, the illustrated rollers 50 have eight recesses 66 each, the recesses having different sizes or electrode configurations. FIG. 14E has deep recesses 66 and FIGS. 14F and 14G have shallow recesses 66. FIG. 14G has triangular electrodes E1,E2. FIG. 14H has a triangular recess 66 with generally rectangular or slightly trapezoidal electrodes E1,E2. FIG. 14I is configured to receive more than one pair of electrodes in each of a plurality of slots 67 of the same recess 66, increasing the opportunity for detection of metals. Electrodes pairs 68 are arranged in groups of three E1,E2,E3, each supported in their respective slots 67,67,67.

In another embodiment, the principles described above can be implemented in modularized equipment and packaged in convenient processing components.

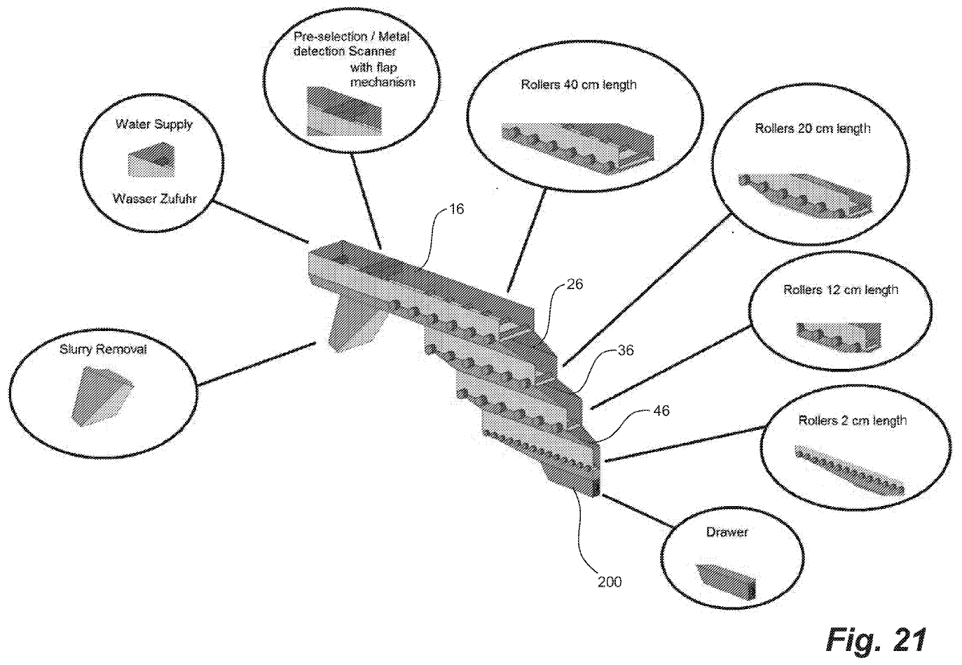

With reference to FIGS. 21 and 22, one form of the system is described having a single unit having four successive stages. A first stage 16 comprises a first channel 14 for receiving the initial feedstream of slurry and having a water supply, a pre-selection metal scanner and a slurry redirection chute fit with a flap for direction of metal-bearing slurry to the electrical detectors, and redirecting non-metal bearing slurry for removal. The first stage 16 also illustrates a plurality of first roller-type samplers, arranged in series along the channel. Slurry, having detected metals therein, is dumped to the second stage 26 for further metals detection. Waste slurry that is not directed to the second stage, is discharged from the first channel 14.

Slurry referred to as waste, either re-directed before sampling, or that which did not get selected by a sampler, or both can be directed to some final processing suited for extraction of trace levels of metals. The stream containment structure of the systems of FIGS. 21 and 22, for removal of waste slurry from each stage, is not shown for clarity of the sampling process.

The second stage 26 comprises a second channel for receiving slurry from the first stage 26 and may or may not also have a water supply. The second stage 26 also illustrates a plurality of second roller-type samplers, arranged in series along the channel. The rollers of the second stage samplers are about one half the diameter of the rollers of the first stage samplers. Slurry, having detected metals therein, is dumped to a third stage 36. Waste slurry that is not directed to the third stage 36, is discharged from the second channel.

The third stage 36 comprises a third channel for receiving slurry from the second stage and may or may not also have a water supply. The third stage 36 also illustrates a plurality of third roller-type samplers, arranged in series along the channel. The rollers of the third stage samplers are again about half the diameter of the rollers of the second stage samplers. Slurry, having detected metals therein, is dumped to a fourth stage 46. Waste slurry that is not directed to the fourth stage, is discharged from the third channel.

The fourth stage 46 comprises a fourth channel for receiving slurry from the third stage and may or may not also have a water supply. The fourth stage also illustrates a plurality of fourth roller-type samplers, arranged in series along the channel. The rollers of the fourth stage samplers are about one fifth the diameter of the rollers of the third stage samplers and can be arranged in a greater density. Slurry, having detected metals therein, is dumped to a recovery bin or drawer 200. Waste slurry that is not directed to the recovery bin is discharged from the fourth channel.

Each stage comprises its own samplers extending transverse to the flow channel. Each successive channel for each successive stage can be narrower as the stream flow rate is reduced and thereby maintain flow velocity and minimize issues such as slurry separation and stagnation. The gravity transition of the sampled slurry to each from an upper stage to the narrower successive lower stage can be physically directed along angled walls therebetween, forming a funnel to direct the stream from a wider upper stage to a narrow lower stage.

As shown in FIG. 22, the system can be modularized lengthwise for ease of assembly and manufacturing. A modular system of sampling units is provided for adding units for parallel stream processing, and for lengthening the channels as needed for processing the feedstream. As shown, all four stages are provided in each module 400 in a vertical manner, and the length of the detection system is increased by adding successive modules 400,400,400 per design or on-site experience. In one example the wide first stage 16, processing downwardly through the second 26, third 36 and fourth 46 narrower stages, can be proportioned at about 40 cm in width, 20, 12 and 2 cm respectively.

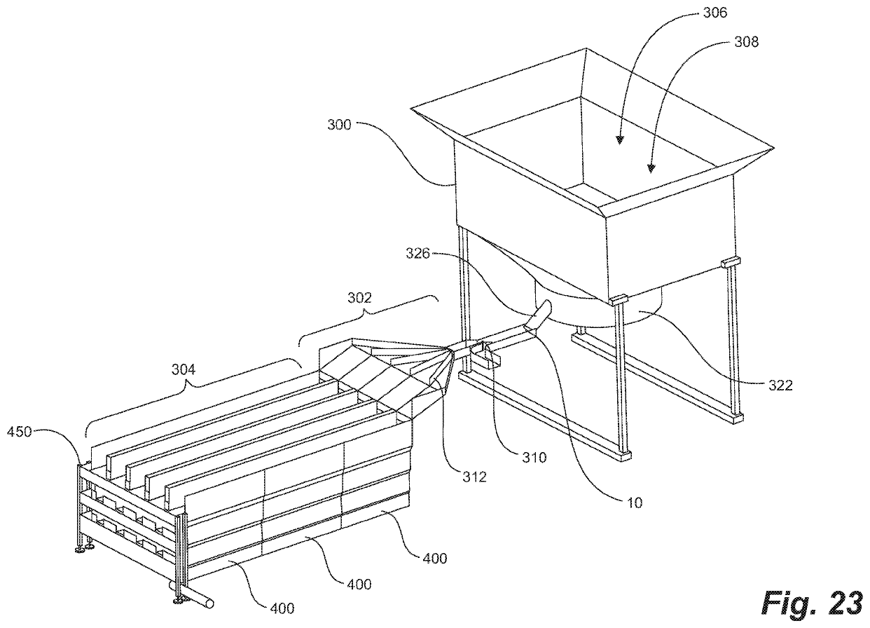

With reference to FIG. 23, another optional selection system is described having a slurry mixing unit 300, a distribution system 302 for parallel units, and a modular system of sampling units 304 for adding units for parallel stream processing, and for lengthening the channels as needed for the feedstream.

As shown, the mixing unit 300 receives stone-sand-ore 306 and rotors mix the stone-sand-ore 306 with water 308 to form the slurry 10. The slurry 10 is directed to the distribution system 302 for metal detection and recovery. Prior to the distribution of the slurry, the slurry passes a metal detector 310 for determining if a sufficient metal content is present to warrant processing by the samplers. If sufficient metals are present, then a fan-like distributor 312 delivers the slurry to each of the parallel sampler units 304. Five parallel units 304A-304E are illustrated. Each illustrated unit 304 comprises three concentrating stages 16, 26, 36 and a recovery tray or bin 200. A waste discharge collector 450 is located at the downstream end of the units 304. The discharge collector comprises a header to receive waste slurry from each unit and combine all streams for transport elsewhere for disposal or final treatment.

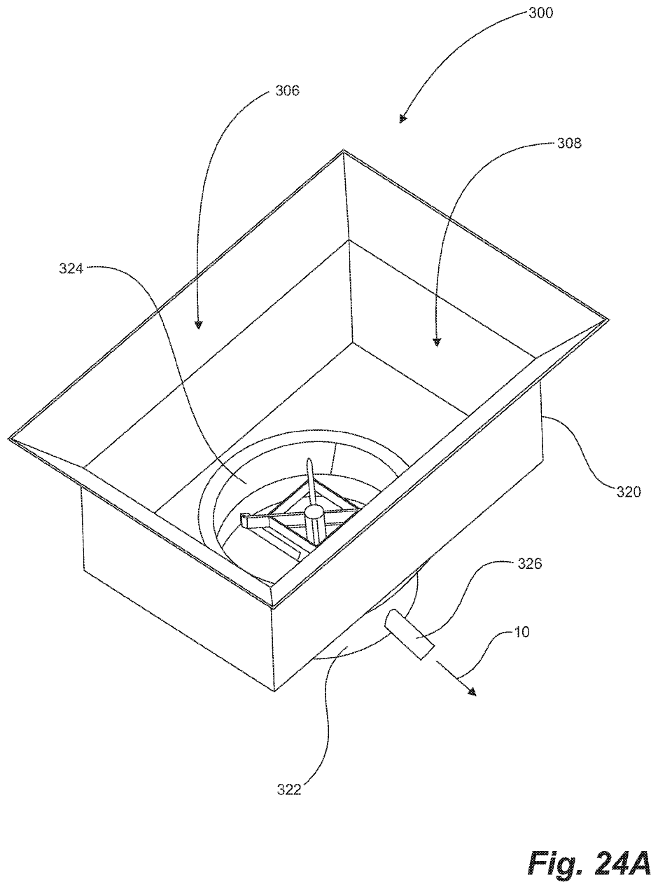

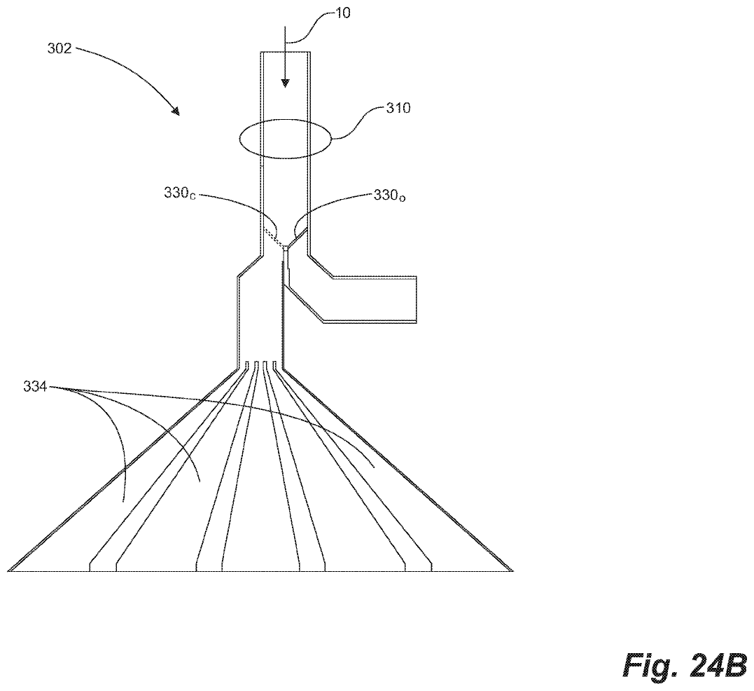

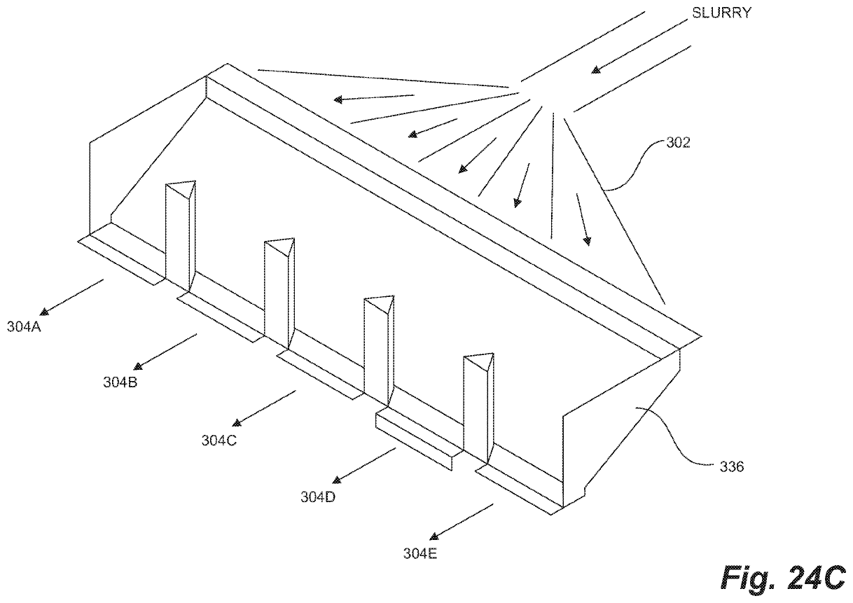

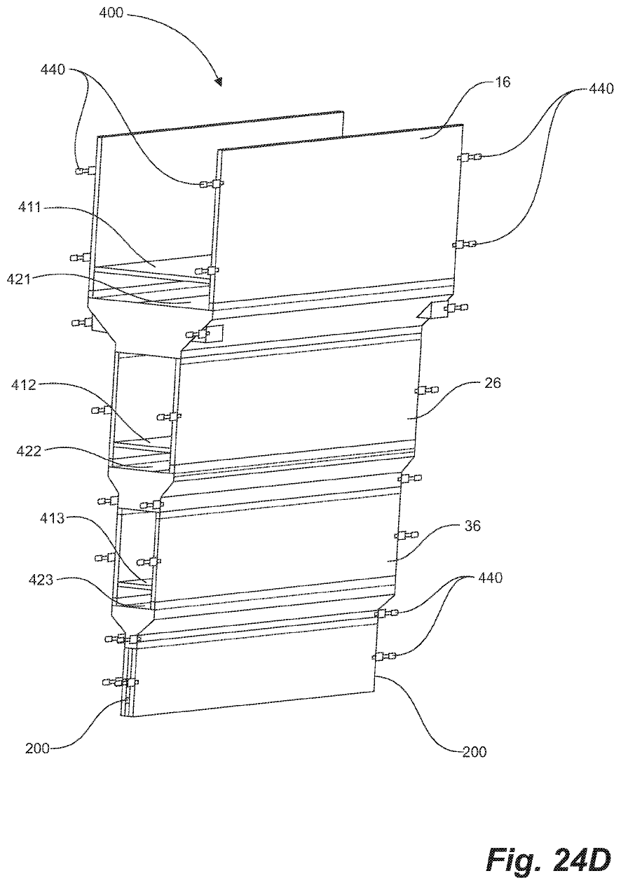

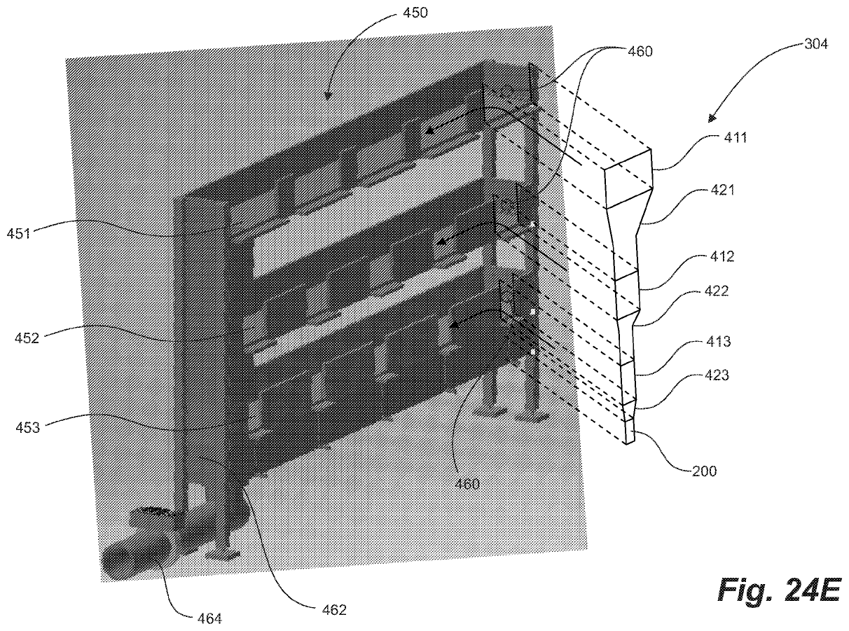

Turning to FIG. 24A, the mixer 300 comprises an open top tank 320 for the receiving stone-sand-ore 306. Water 308 can be added from a variety of locations or pre-mingled with the stone-sand-ore 306 being added to the tank 320. The bottom 320 of the tank is cylindrical, having a vertical axis and one or more mixing blades or rotors 324 rotatable about the axis for mixing the ore and water in a vortex to form the slurry 10. In an embodiment, two rotors 324 are provided having different diameters. The slurry exits via a bottom discharge 326.

As shown in FIGS. 23,24B and 24C, the slurry 10 is discharged to the distribution system 302 for initially determining if the slurry should be directed to the samplers or redirected to waste. If the metals content does meet a threshold, the slurry 10 is distributed to each parallel unit 304 of the multi-unit selection system. As stated above, the slurry 10 is initially scanned, such as at detector 310, for a sufficient amount of metal therein. This is a binary condition; either there are sufficient metals in the slurry to meet a threshold level, or not. An example threshold might be in the order of 9 gm of metal per tonne of mined ore. As this process is a materials handling system, having erosive materials, the samplers 18 of the selection system are subject to needless less wear and tear if the slurry does not contain a commercial threshold of metal.

If the initial metal scan does not meet the threshold, a gate 330 remains in the redirection position 330c to redirect the slurry to waste or further processing. If the initial reading from the initial scan meets or exceeds the threshold, a positive signal is generated and an actuator moves the gate 330 to the selection position 330o to direct the slurry to the samplers of the first stage of the five unit metal selecting system.

The slurry is physically split into five streams along five chutes 334. All five streams 334 are discharged into a laterally extending common distributor tank 336. As shown in FIG. 24C, the common tank 336 permits some liquid re-equilibrium between the split streams of slurry 10 before entering the first stages 304A through 304E of each unit.

With reference to FIG. 24D, and as described above, each stage performs its sampling, selection and collection of metal-bearing slurries. In this example there are three stages 16, 26, 36, shown with a recovery tray 200 therebelow.

In this embodiment, the units 304 are assembled from like building modules 400. Modules can be assembled end-to-end for adding additional samplers for each stage. As shown in FIG. 23, three modules 400,400,400 are assembled end-to-end for lengthening the feed channels and, further, the triple-modules are duplicated a five, side-by-side parallel units 304A through 304E for increasing throughput. Each vertically arranged stage 16, 26, 36, has a feed channel 411,421,431 and collection channel 412,422,432 respectively.

The modules 400 can be latched or secured together end-to-end and side-to-side using over center latches 440 or other suitable connections. Each latch 440 can include an alignment pin to engage between modules, a fixed latch, and a movable drawbar that engages the fixed latch and pulls two modules together. The module interface, at the bottom of each channel, can include an elongated bar and slot arrangement to form a liquid barrier to minimize fluid loss at those interfaces. For long-term installations, one can further secure the modules together by welding or the like. Further, each stage within a module can be coupled to an adjacent stage using a tongue and groove engagement for adding and removing stages depending on the site.

As shown in FIG. 24E, each unit 305 can be assembled from selector module 400. As shown assembled in FIG. 23 and as one individual; component in FIG. 24D, the individual modules 400 for the embodiment of FIG. 23 can comprise all three stages 16, 26, 36 and the recovery tray 200. The individual selector module is manufactured in manageable lengths that can be connected end-to-end to form a length needed to sample and select metals from the given slurry flow parameters. As shown in FIG. 23, three selector modules 400 are connected together end-to-end and five assembled units, of three selector modules each, are connected side-by-side to form the five parallel units 304.

Each individual selector module 400 is sized for ease of handling, and constructed to be self-supporting to maintain the channels and support the samplers.

End connectors can be of the draw latch 440 form to connect then draw the adjacent ends of modules together, before over-centering to securely lock the connection together.

Locating pins and alignment holes (not detailed) at abutting faces can be provided to ensure aligned liquid interfaces are formed.

As shown in FIG. 23 and FIG. 24E the flow stream channels of each stage terminate at a discharge collector 450. The collector 450 receives and combines the waste or low-metal bearing slurry that was not selected. The collector 450 is a multi-tiered vessel, each tier 451,452,453 corresponding to stage 16,26,36.

Water jets 460 are provided on every tier 451,452,453 and which aid to direct the collected slurry to a drain 462. The drain flows to a bottom outlet 464 and waste slurry is discharged therefrom. The waste slurry may undergo one final detection and sampling before removal to tailings.

In embodiments, various features and advantages are achieved. A process is provided for selecting metals especially precious metals comprising first crushing the earth rock containing gold, silver and other metals to a size from 1 cm=10 mm to 0.5 mm. The size of detected metals can be adjusted by adjusting the size and spacing of the electrodes. The ore is crushed to a size or dimension of at least as large of about a spacing of the gap between the pair of electrodes. The crushing process exposes the metal for contact with the electrodes and detection. Depending on the ore and metals therein, the skilled mining personnel can adjust the crushing process including selecting the type of mill. Metals in sampled slurry are identified in the recess slurry by a small current generated by a low potential across the electrodes, such as voltages of under 1 Volt across the contacts. A controller generating an electrical signal upon detecting metals at the electrodes and rotates the roller to temporarily align the recess and detected metals to dump the sampled slurry into an opening beneath the roller whereby metals are recovered from the sampled slurry and the balance is passed as waste slurry over the roller.

The process can be repeated by sampling the slurry in a subsequent passing, identifying and recovering process. The repeating the passing, identifying and recovering process for the sampled slurry in subsequent processes until a high concentration of required metals remain.

The process can include a channel with an opening beneath the roller that has a closable opening integrated into the channel or with an electrical contact or signal for controlling the opening function, either electrically or electronically. The channel further comprising an angled opening or recess which extends from side to side across the channel is opened and closed by a flap for bypassing the detection apparatus if no metals of interest are present in the ore.

For a coarse determination of the presence of metals in the slurry, the selection system comprises at least one pair of electrical powered contacts or other metal detector in proximity or in contact with the slurry. The initial detector generates a signal once a threshold amount of the metal of interest, such as gold, silver or platinum particles, are detected. The signal is indicative of the identification of said metals, the signal actuating a step motor or other actuating mechanism for opening the passage of slurry to the metals sampling and selection area.

Each of the one or more rollers has electrical contacts in the sample recess and generates a signal once detecting gold or silver. The signal activates a step motor which turns the roller to empty or dump the recess. The recess is rotated from above the channel to below the channel to dump a metal-containing sample, and then return to the sampling position. A recess could be rotated one direction to the dump position and back the other direction to the sampling position. One could rotate continuously one direction to the dump position and through to reset to the sampling position. With multiple recesses one could rotate or rotationally index in the same direction to sequentially place one recess in a dumping position while another recess is already in the sampling position.

In another embodiment, rather than a recess, a flap in the channel can be opened briefly, by mechanically or electrically means, to open an opening in the channel.

One form of detector includes metal detector bars or electrodes of rectangular cross section placed at an angle of 90° to each other.

The electrodes can comprise two metal bars that identify metals once being in electrical contact with the desired metal.

The sampler can be a roller fit with one or more recessed, each recess supporting electrical contacts which give a signal to a step motor, a reading unit position, and a micro-controller which is connected to a driver/software unit and a power supply. The turning roller has sliding electrical contacts to connect the signal made by the electrodes to the controller and back to the step motor.

The rollers are sealed off on the ends to minimize access of dirt and corrosion to the bearings.

The recess and roller generally can be periodically or constantly sprayed with high-pressure water jets, and maintenance of the roller is readily achieved by separating a top bearing housing at each end, or the rollers can be secured by a spring “release” mechanism.

The sampling recess in the roller can be arranged to select a portion of the slurry and avoid rejecting slurry by high speed rotary movement.

The rollers can be cleaned by water jets which wash accumulated, dried, or otherwise non-productive slurry out of the sampling recess and from between the rollers and channel.

In another embodiment, as shown in in-situ in FIGS. 23 and 24D, the structure of the modular components walls can be formed of molded or machined components, such as thermoplastic polyester including polyethylene terephthalate (PET-P) or other plastics, plastics being suitable on the basis of mechanical strength, economy, low weight and for precision in both interconnections and support and alignment of samplers 18. As discussed previously the system can be modularized with a series of modular sampling units 304, in series for processing length, side-by-side parallel units for capacity and stacked stages for staged refining.

Modular sampling units enable compact shipping of many components to a processing site. In an embodiment, one system has 180 modular sampling units 304 comprising three parallel (side-by-side) sets of ten units in series (end-to-end) for each of six stages (stacked one above the other). In other words, there are 30 identical units in stage one, and 30 identical units for stage 2 and so on. This would be a significant bulk, of mostly empty space and awkward for shipping, except for the disclosed modular design having added capability for shipment in disassembly flat packs.

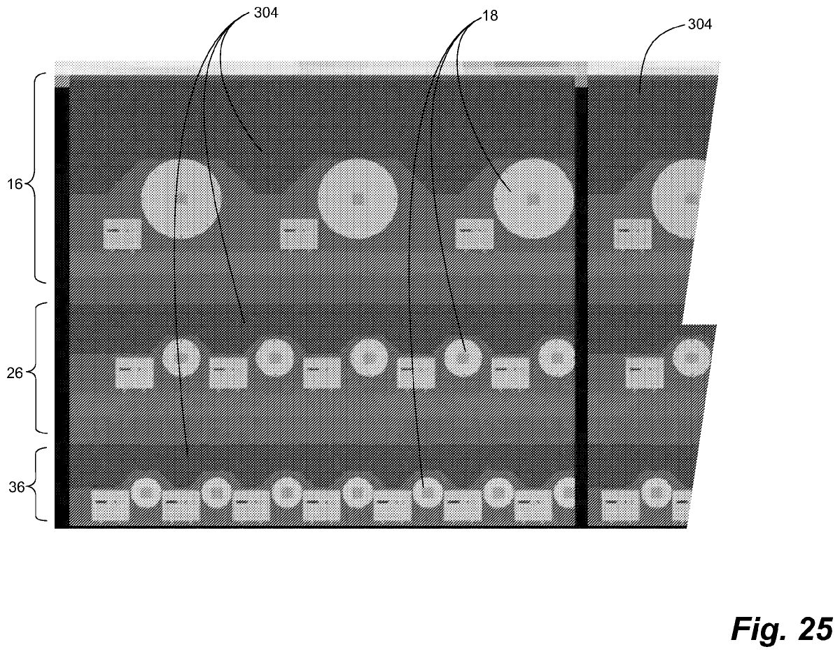

In FIG. 25, a three-stage 16,26,36 section is shown for one and a partial section of the units 304,304 in series. Each modular sampling unit 304 for a stage can be identical, the particular stage dictating the size and numbers of samplers 18.

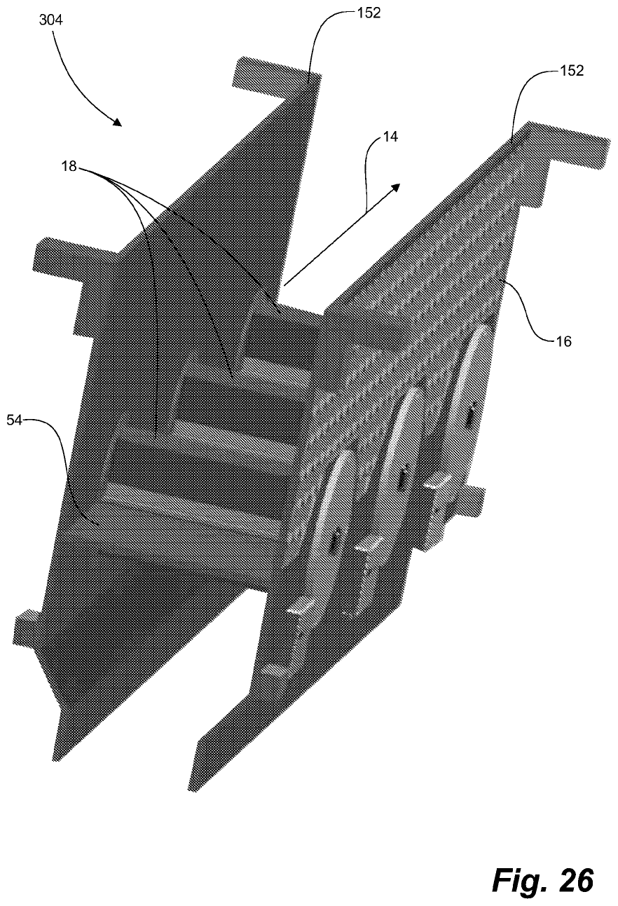

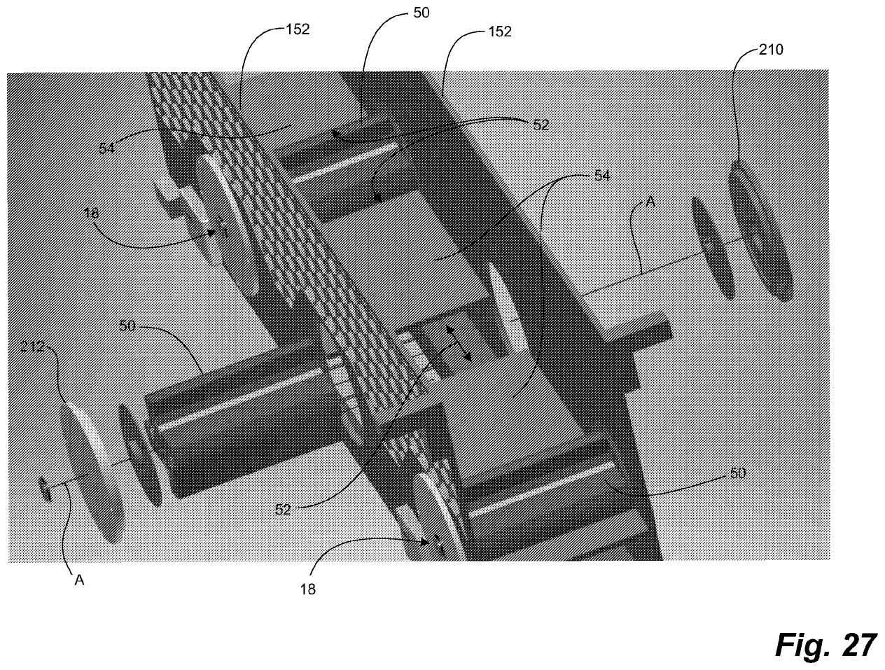

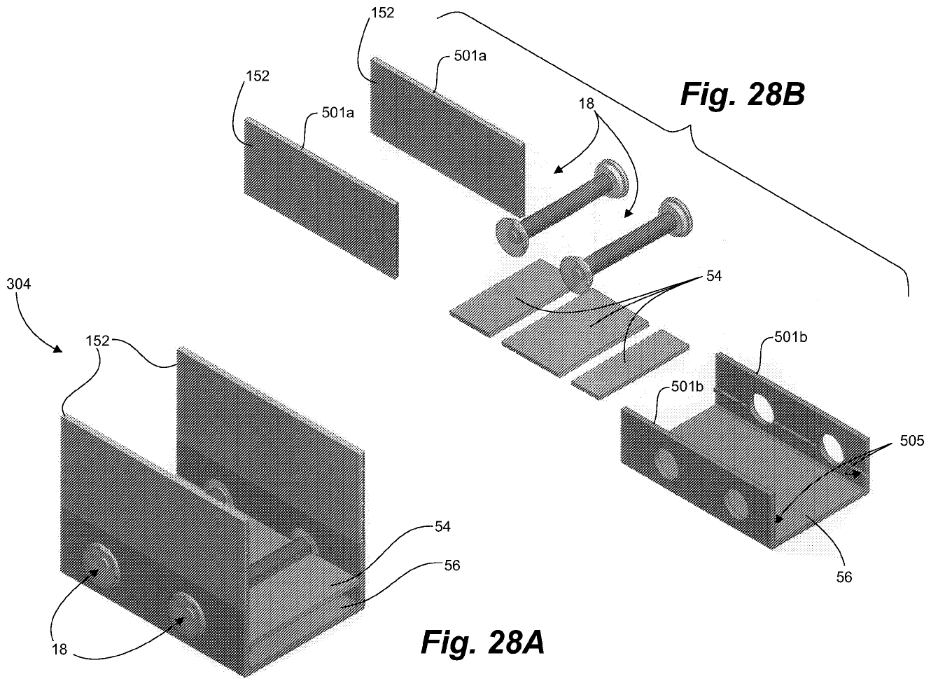

As shown in FIG. 26, each the modular sampling unit 304 can be designed as having three samplers 18,18,18, in this case acting as the first stage 16 for initially receiving the stream of slurry. The modular sampling unit 304 comprises opposing side walls 152,152 of the channel 14, the bottom plate 54 of the channel, one or more samplers 18, and recovery tray or subsequent stage below. A next stage, having a different-sized, usually smaller modular sampling unit, couples to the bottom of the recovery tray of the upper stage's unit. In FIG. 28, one sampler of the unit of FIG. 26 is shown in exploded view illustrating the sampler-receiving slot 52 and attachment arrangement for sampler end plates 212,10. The sampler's end plates 212,210 determine the axis A and alignment of the roller 50 with the slots 52,52,52 between the channel's bottom plates 54.

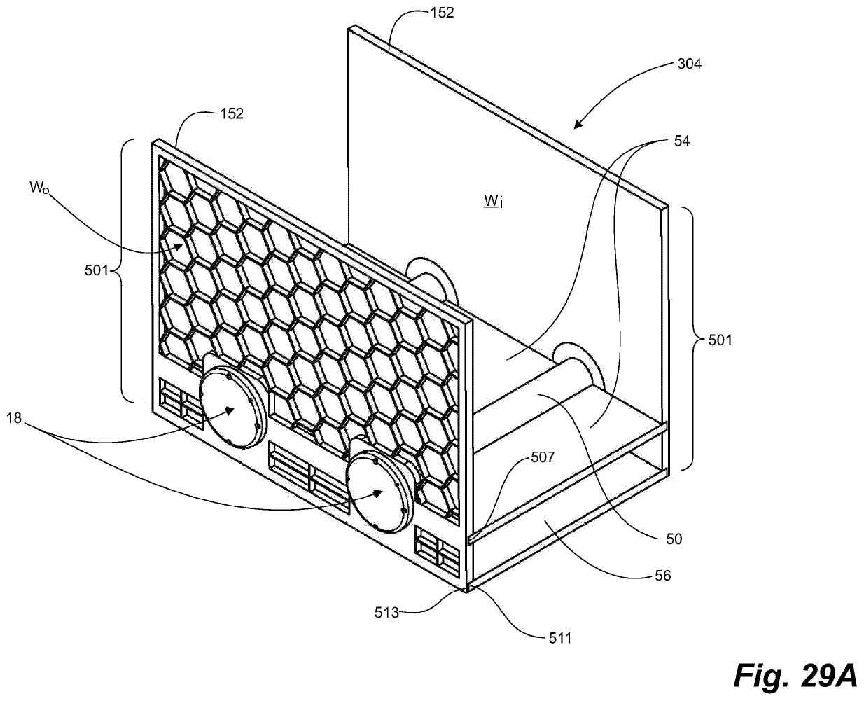

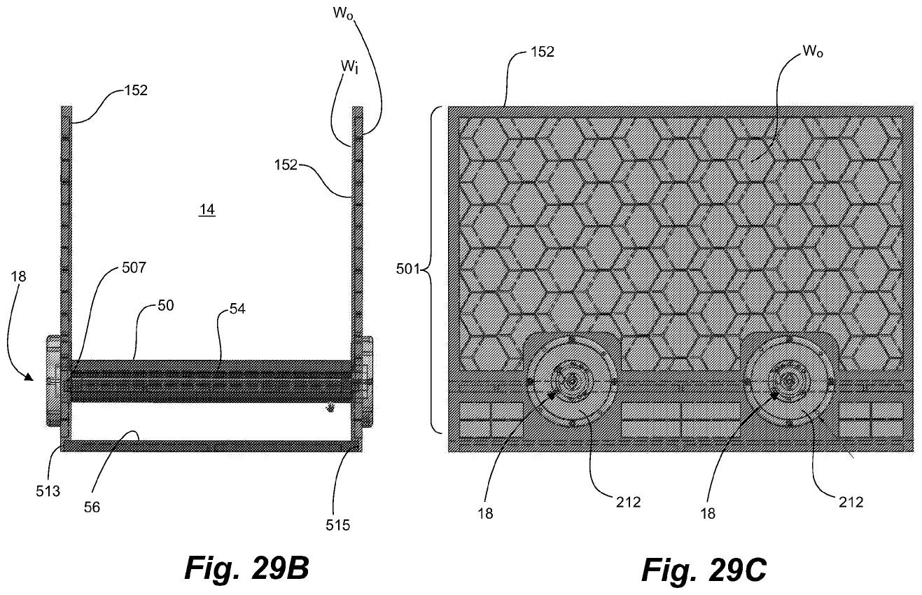

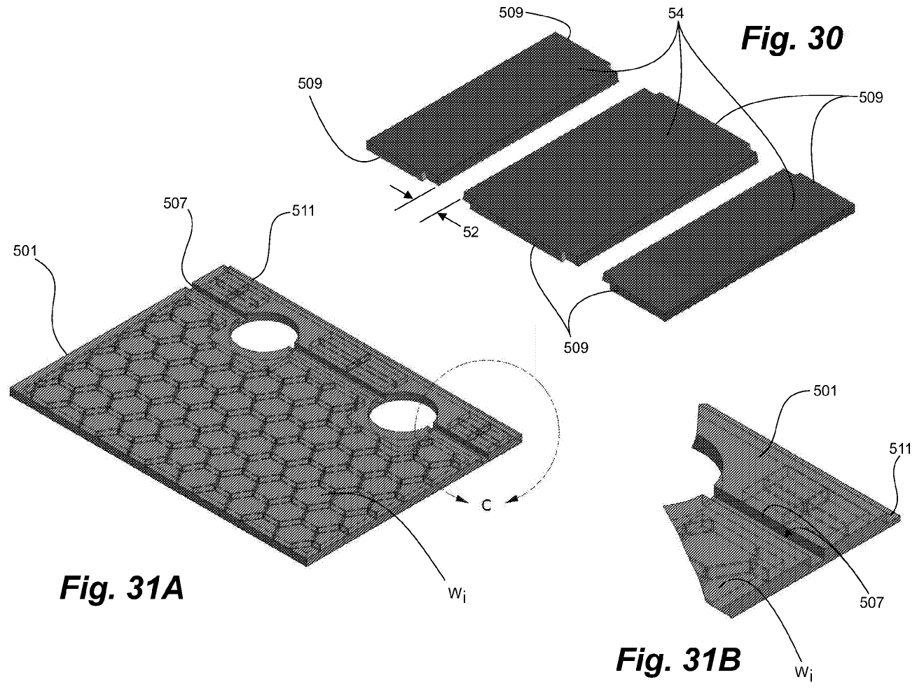

Illustrated herein, each side wall panel 152,152 can be assembled of several sub-panels 501a,501b per FIGS. 28A and 28B, or be a unitary structure of one panel 501 as shown in FIGS. 29A,31A. Manufacturing challenges using one piece panels 501 for each side wall 152 can be offset with improved repeatability I manufacture and precision mounting of the samplers 19 relative to the straddling bottom plates 54,54.

With reference to FIGS. 29A and 29B, each side wall 152 is a two-component panel system with a lower U-shaped base 503 for receiving and supporting the samplers 18, the base 503 being fit with opposing ledges 505,505 for receiving and supporting the bottom plates 54,54,54 of the fluid channel 14. The base forms lower side panels 501b,501b. Upper side panels 501a,501a are separate from the base 503 and secured to lower side panels 501b,501b of the U-shaped base to form the side walls 152,152. Care is taken to ensure the bottom plates 54 are secured to the ledges for vertical and horizontal alignment with the sampler 18 for minimizing sample loss through slot 52.

In the case as shown in FIGS. 29A, 29B and 29C, in another embodiment of a modular sampling unit 304, each side wall 152 can be a unitary planer rectangular side panel 501. Two samplers 18,18 are shown, the modular sampling unit having a pair of opposing side walls 152,152 each formed of a unitary panel 501. The bottom plates 54 of this unit are formed of three individual bottom panels forming two sampler slots 52,52 therebetween and a one-piece recovery tray 56. In this case the upstanding portion of panel 501 includes a hexagonal structure as illustrated, generally understood as having excellent weight and mechanical properties. The bottom panels can also be formed of plastic panels, such honeycomb strengthened panels, the upper surface being featureless for the flow of slurry or collected samples therealong.

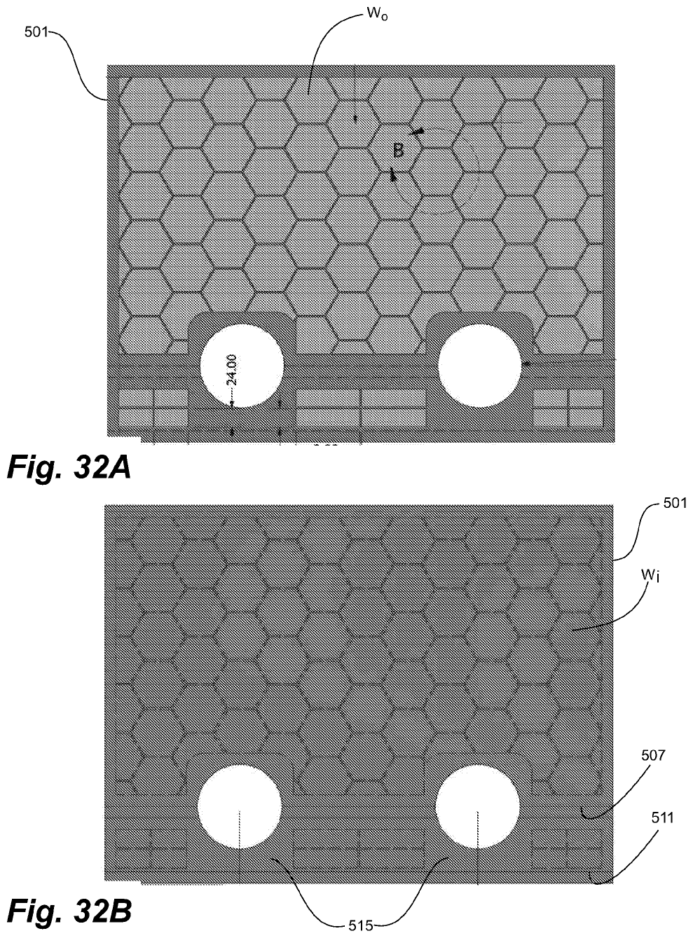

With reference to FIG. 29B and FIGS. 31A and 31B, an inside Wi of each panel 501 is equipped with at least a first floor assembly groove 507 extending longitudinally along the panel to receive opposing linear side edges 509 of each of one or more bottom plates 54. The inside Wi the panel 501 is otherwise clear of features for minimizing disruption and trapping of slurry. The outside Wo of the panel 501 can have the honeycomb structure exposed as it is not exposed to slurry. A second floor assembly groove or recess 511, spaced downward from the first groove 507, can receive opposing linear edges 513 of the plate 56 of the recovery tray. As shown in FIGS. 29C and 32A, the opposing side, or outside Wo, of the panel 501 can have alignment recesses 515 for locating and receiving the sampler mounting structure, such as plates 210, 212. The outside Wo of the panel 501 can also have other templates or mounting locations for controllers and other components.

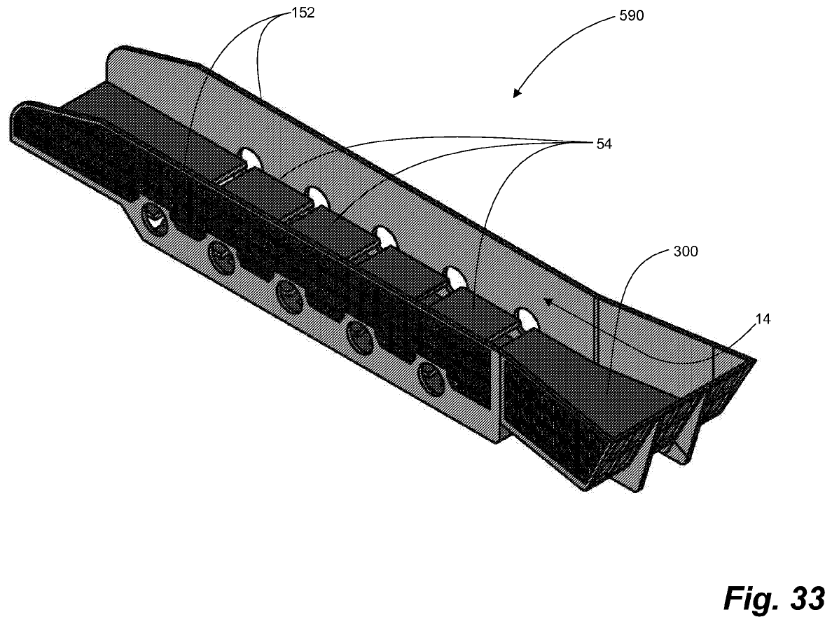

Turning to FIG. 33, a compact, small operation system 590 can be embodied in a complete unit. As shown, the compact system 590 includes two elongated side walls forming a channel therebetween, coupled with a hopper 300. The system 600 is shown fit for five samplers, and bottom plates 54 and a recovery tray 56 therebelow. The samplers are removed from the system for simplicity. The system can be sized for shipment assembled to the site and is suitable for small operations.

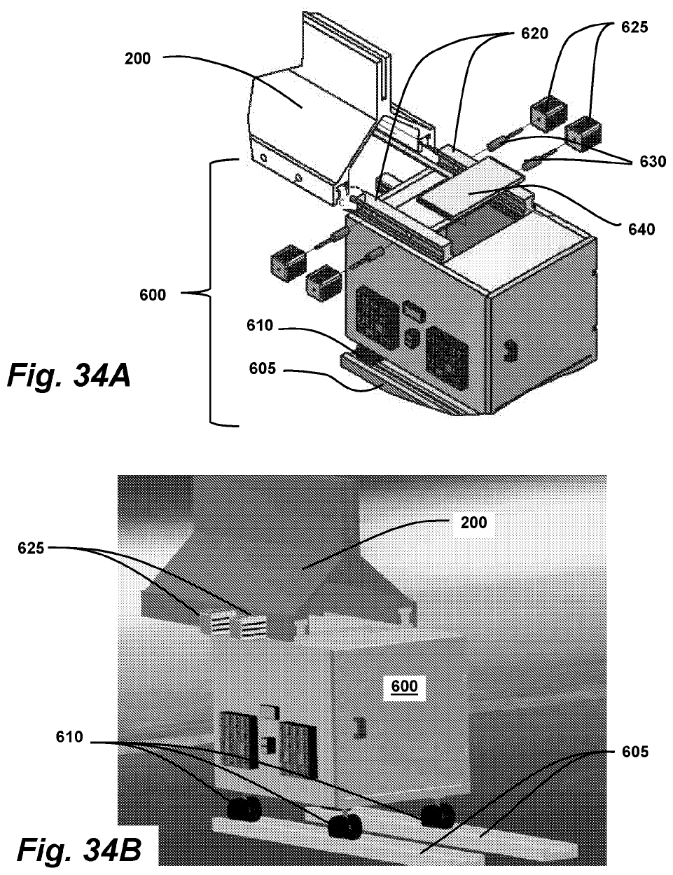

In FIGS. 34A through 39B, an automated security device or box is provided for final filtration, collection and removal of the collected precious metals. As mining is often an automated process, and particularly with the modular system disclosed above, regular and also automated collection and recovery of the metals is provided. As the above system results in highly concentrated forms of the selected metal for recovery, the security of the end product is warranted.

Generally, a secured box is locked to each recovery tray of the apparatus. Other embodiments may converge multiple streams of collected metals from multiple parallel recovery trays. The box is locked to the apparatus until deemed ready for recovery, either by an operator, or my sensors.

Presentation of appropriate security credentials enable unlocking of the box for transport to a recovery and storage facility. There are several options for triggering recovery and replacement of a filled box with an empty box including: an authorized individual noting the fill level and entering a security code or biometric identification, or sensors indication a filled state for automatic de-coupling and transport to storage. The box can be on rails or fully robotic for self-guided transport to storage.

With reference to FIGS. 34A and 34B, a security box 602 is shown de-coupled from the final recovery tray 200, the box being shown in exploded form for identification of key security components. The box comprises a secure enclosure, resistant to tampering, and, in this embodiment, the box is supported on wheels 610 that engage tracks or rails 605. The top of the box includes an access panel 640 and a T-slot locking system 620. The access panel 640 is operable between an open collection position and a closed transport position.

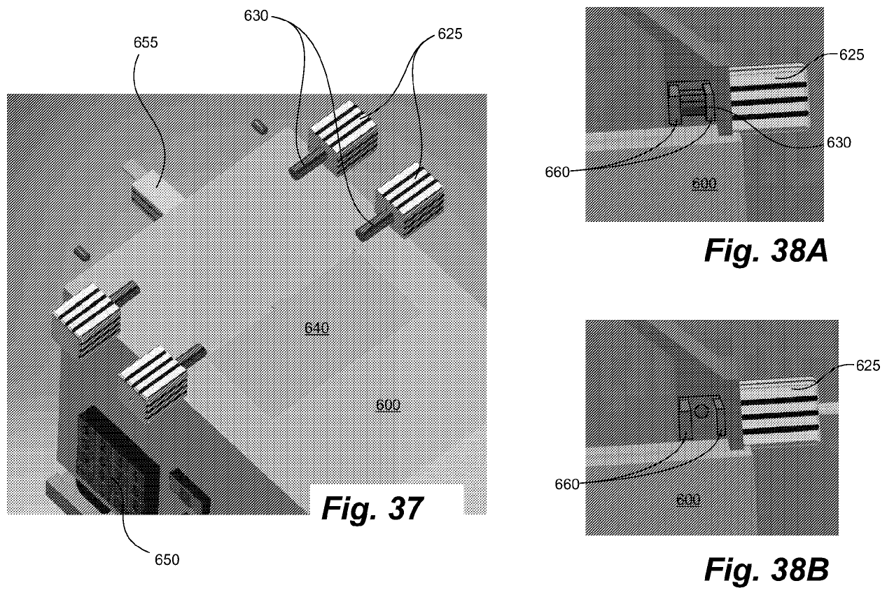

The T-slot locking system 620 comprises two interlocking components: a T-rail shown here attached to the box 600 and a T-slot shown formed at the bottom of the recovery tray 200. Locking actuator 625 lock the T-slot and T-rail together. Also shown in FIG. 37, hydraulic or other locking actuators 625, secured to the tray housing and T-slot, drive a bolt 630 through a hole in the T-rail to lock the T-rail and T-slot together. The bolt is supported on either side of the T-rail by bosses 660. The bottom of the tray 200 is a housing or peaked roof system for forming a cover over the box 600 when locked thereto.

As shown in FIG. 34B, the tray 200 and box 600 are coupled. The panel 640 is open for collection of precious metals. The recovery tray 200 has an opening therein arranged directly over the panel 640. Concentrated metals from the recovery tray 200 fall past open panel 640 for deposit into the box 600.

Sensors including metal detection can advise the presence of precious metals and using time-based analysis, can analysis the rate or quantity of metals collected. Depending on infrastructure and utilities, the security box 600 can be connected to the internet for real-time or batch uploading of collection data, such as to the operator's head office. In remote locations, satellite terminals can be provided and operated on solar or other replaceable, renewable or rechargeable power supplied. Satellite terminals are suitable for SCADA or other data communication and batch or continuous data upload to a central processing center.

Alternatively, load sensors in the box supports or in a weigh scale under the box can provide continuous data on collections, depending on the size of the installation can be in the kilograms to tens of kilograms.

Selected and collected material including concentrated metals falls into the box and onto a filter screen 645 in the box 600. The screen 645 can supported on vibrating support and water can be flushed across the screen to aid in separation of the precious metals which pass through the screen from the balance of the slurry which is washed off the screen. Under the screen, one or more sensors indicate the height of the collected metals. Flushing water can be drained from the box.

The aforementioned weigh scale, or level sensors can be used to signal the box is filled. A pair of contacts can be activated when the depth of the metals reaches the contacts. When a pre-determined depth or weight is reached, equivalent to a filled level, as signal is generated to for exchanging the box with an empty box.

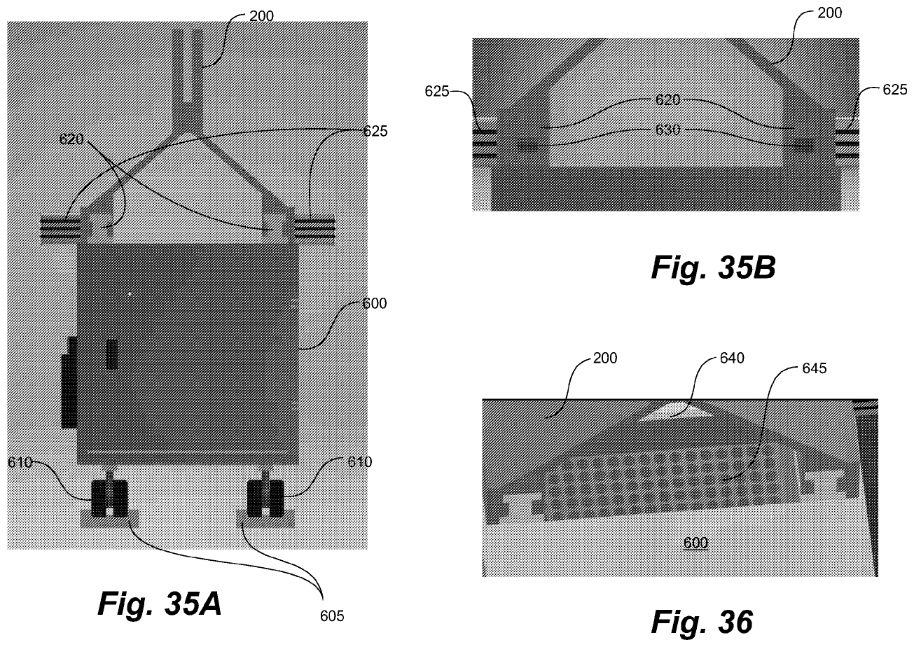

When ready for removal and storage the box 600 is locked and de-coupled from the recovery tray 200. With reference also to FIGS. 35A and 35B, the T-rail is aligned with the rails 605, so that when unlocked, the box can be moved co-axially along the rails, from underneath the recovery tray 200, when the T-rail and box moves co-axially along the T-slot, eventually de-coupling the T-rail and T-slot. As shown in FIG. 38A the actuator 625 and locking bolt 660 are engaged to lock the tray 200 to the T-rail. As shown in FIG. 38B, when signaled to unlock the actuator 625, locking bolt 660 is retracted to disengage from the T-rail to unlock the tray 200 therefrom.

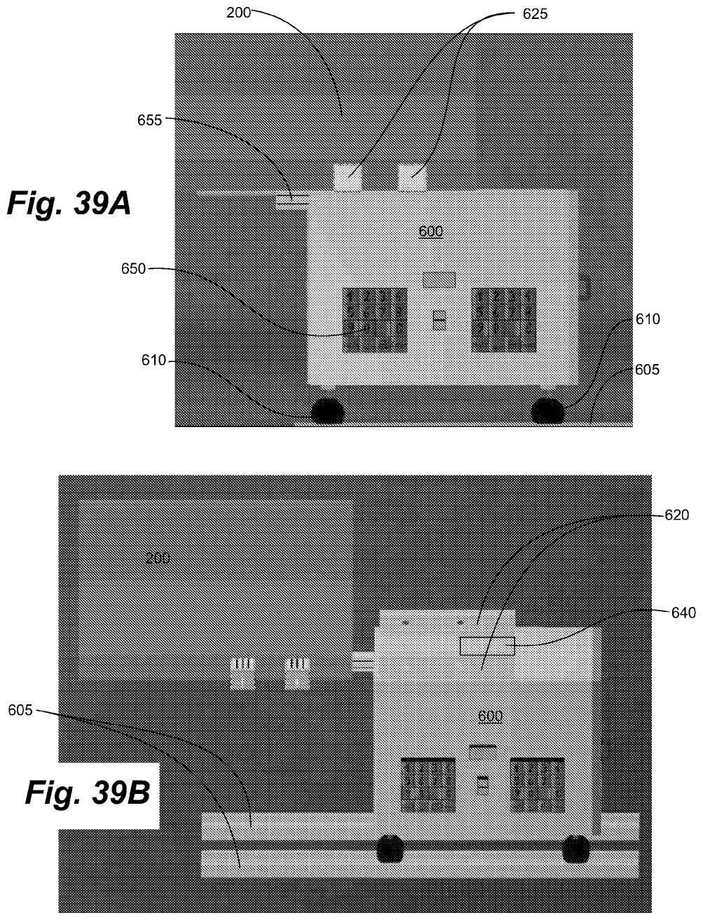

With reference to FIG. 39A, the security box is shown collecting metals. When filled, or some other pre-determined threshold is triggered, the securing and removal of the box 600 is initialed. An authorized personnel, or automated release, determined that the box should be release. The authorized individual or personnel can enter a security code on panel 650, or other biometric authorization is provided including fingerprint or retinal scan.

In any event, if authorized in person, or automatically, the box is secured before release from the recovery tray. The recovery tray 200 is temporarily blocked with a tray door (not shown) to store collected metals until a replacement box is in place. The panel 640 closes, and the T-slot locking system is dis-engaged. Once dis-engaged, the box 600 can be manually rolled out from under the recovery tray 200, or automated transport systems can be engaged to drive the box to a storage area for secure unloading and cleaning as necessary.

A replacement box is provided and moved, such as by the same manual or automated transport system, to couple with and be locked to the recovery tray 200. When locked, the box panel 640 can be opened, and tray door can be opened to discharge staged collected materials and ongoing collected materials in the box 600 to begin the cycle once again.

We claim:

1. A process for selecting metals of interest from ore comprising:
selecting a sample of a first feed stream of slurry of ore and water flowing across at least one electrical detector for establishing detector signals indicative of the presence of the metals within the slurry sample;
upon receiving the detector signal, then
directing the slurry sample to a second collection stream containing the metals; and
recovering metals collected from the second stream.

2. The process of claim 1, wherein each electrical detector comprises a pair of electrodes, the process further comprising crushing the ore to a size of at least about a gap between the pair of electrodes.

3. The process of claim 1 further comprising:
one or more samplers wherein each sampler incorporates at least one of the one or more the electrical detectors;
flowing the first stream across each sampler and upon receiving the respective electrical detector's signal;
directing the slurry sample selected for that sampler to the second collected stream; and
flowing a balance of the first stream of slurry as an overflow feed stream of slurry to a subsequent sampler of the one or more samplers.

4. The process of claim 3, wherein before flowing the first feed stream over the one or more samplers further comprising detecting metals in the first feed stream and, if a concentration of metals is below a threshold level, redirecting the first feed stream to a waste stream.

5. The process of claim 3 further comprising extending each sampler transverse to the first feed stream, the at least one electrical detector extending along the sampler, wherein the directing of the slurry sample to the second collected stream further comprises actuating the sampler to separate the sample from the first feed stream and deposit the slurry sample into the second collected stream.

6. The process of claim 5, wherein the one or more samplers are in series and flowing the first feed stream across each sampler flows the first feed stream across each sampler in the series.

7. The process of claim 5, wherein:
each sampler is a roller, each electrical detector is housed in a recess formed longitudinally along at least a portion of the roller, and
actuating the sampler to separate the sampled slurry from the feed stream further comprises rotating the roller to separate the recess and sampled slurry captured therein from the first stream and direct the sampled slurry, and metals therein, to the second stream.

8. The process of claim 1, further comprising crushing the ore and mixing the ore with water for forming the first stream of slurry.

9. The process of claim 1, wherein:
the selecting a slurry sample from the first feed stream and collecting the second collected stream forms a first stage of selecting metals and
the second collected stream forms the first feed stream for a successive stage of selection.

10. The process of claim 9, further comprising the first stage and at least one successive stage of selecting metals, wherein the successive collected stream from a last of the successive stages of selecting metals forms a recovered metals stream.

11. The process of claim 10, wherein the first stage and the at least one successive stage of selecting metals are processed in a module, and a length of the first feed streams of each stage is lengthened by coupling additional modules end-to-end thereto.

12. The process of claim 10, wherein the first stage and the at least one successive stage of selecting metals are processed in a module, and the throughput of the process is increased by coupling additional modules side-by-side thereto.

13. The process of claim 10, further comprising:
depositing the recovered metals stream into a security box; and
when full, replacing the full security box with an empty box.

14. Apparatus for selecting metals from a slurry feed stream comprising
at least one sampler for receiving at least a slurry sample from the feed stream;
at least one electrical detector for establishing detector signals indicative of the presence of metals within the sampled slurry; and
an actuator for operating the sampler upon receiving the detector signal to direct the slurry sample to a collected stream.

15. The apparatus of claim 14 further comprising, a channel for directing the feed stream to the at least one sampler.

16. The apparatus of claim 14 further comprising:
a metal detector upstream of the at least one sampler for establishing dump signals indicative of the absence of presence of metals therein;
a diverter; and
upon receiving dump signals, actuating the diverter from a sampling position to a dump position for dumping the feed stream to a waste stream.

17. The apparatus of claim 16, wherein upon cessation of the receipt of dump signals, actuating the diverter from the dump position to the sampling position.

18. The apparatus of claim 14, wherein the at least one sampler comprises two or more samplers arranged in series along the channel.

19. The apparatus of claim 14, wherein:
the feed stream is directed along a feed channel and the collected stream is directed along a collected sample channel, the feed channel, at least one sampler and the collected sample channel forming a first stage for the selecting of metals, and
the collected sample channel forms the feed channel for a successive stage.

20. The apparatus of claim 19, wherein the successive collected stream from a last of the successive stages of selecting metals forms a recovered metals stream, further comprising
a recovery tray for receiving the recovered metals stream; and
a security box removably locked to the recovery tray for receiving recovered metals from the recovery tray; and
when the security box full, unlocking the security box from the recovery tray.

21. The apparatus of claim 19, wherein the first stage and the at least one successive stage of selecting metals are housed in a module, further comprising two or more modules arranged end-to-end for lengthening the feed channel.

22. The apparatus of claim 19, wherein the first stage and the at least one successive stage of selecting metals are housed in a module, further comprising two or more modules arranged side-by-side for increasing the throughput of the apparatus.

23. The apparatus of claim 19, wherein the feed channel comprises side walls and a bottom plate, the bottom plate having one or more slots extending transversely thereacross for receiving each of the at least one sampler.

24. The apparatus of claim 23, wherein each of the first stage or each of the successive stages are housed in one or more like modules for that stage, each module connectable with another of the like modules.

25. The apparatus of claim 24, wherein each module comprises a pair of unitary side panels for forming the side walls and two or more bottom panels forming the bottom plates, the bottom panels affixed at their longitudinal edges to longitudinal assembly grooves in each side panel for spacing the side panels apart to form the channel, the assembly grooves aligned with the sampler axes.

26. The apparatus of claim 19 the feed channels of each stage terminate at a discharge collector for combining a waste stream of slurry.
